(19) United States
(12) Patent Application Publication
(10) Pub. No.: US 2015/0175712 A1
(43) Pub. Date: Jun. 25, 2015

(54) ANTAGONIST TO AN ENZYME AND/OR A METABOLITE OF THE KYNURENINE PATHWAY

(71) Applicant: ImmuSmol SAS, Pessac (FR)

(72) Inventor: Alban Bessède, Bordeaux (FR)

(73) Assignee: ImmuSmol SAS, Pessac (FR)

(21) Appl. No.: 14/409,330

(22) PCT Filed: Jun. 21, 2013
(86) PCT No.: PCT/EP2013/063070

§ 371 Date: Dec. 18, 2014

Related U.S. Application Data
(60) Provisional application No. 61/662,419, filed on Jun. 21, 2012.

(30) Foreign Application Priority Data
Jun. 22, 2012 (GB) 1211120.9

(51) Int. Cl. C07K 16/44; C12Q 1/68; C07K 16/40; C12N 15/115; A61K 45/06; A61K 39/395; G01N 33/574

(52) U.S. Cl. CPC C07K 16/44; A61K 39/39583; A61K 39/3955; C12Q 1/6886; G01N 33/57488; C12N 15/115; A61K 45/06; C07K 16/40; C07K 2317/76; C12N 2310/16

(57) ABSTRACT

The present invention relates to a modulator of an enzyme and/or a metabolite of the kynurenine pathway (FIG. 18).

### FIELD OF THE INVENTION

The present invention is related to an antagonist to an enzyme and/or a metabolite of the kynurenine pathway.

### BACKGROUND

### The Tumor Immune Escape

Tumor progression implicates several factor named as “hallmarks of cancer” (Hanahan et al 2011) Among them, resistance to cell death, exaggerated proliferation, genomic instability, and tumor angiogenesis are factors that promote tumor growth. However, tumor immune escape is a key factor in cancer progression, which has been neglected for long time. When a cell becomes malignant by transformation, i.e., when it becomes different from the “self”, it should be recognized and eliminated by the immune system. Accordingly, transformed cells are continuously eliminated in our organism by a functional immune system. Unfortunately, a tumor cell is, in some cases, able to repress the immune response. This process is called the “immune escape” or “tumor escape” (Dunn et al 2002) and is partially controlled by the indoleamine 2,3 dioxygenase (IDO1)/tryptophan 2,3 dioxygenase (TDO2) enzymes (Prendergast 2008; Opitz 2011).

### IDO1 and TDO2 as a Therapeutic Target in Oncology

Based on these findings many researchers have proposed pharmacological inhibition of IDO1 as a new treatment in oncology. Several studies showed that IDO1 inhibition by 1-Methyl Tryptophan (1MT), the prototypical IDO1/2 inhibitor, possesses anti-tumor properties. However, when administered alone, benefits of 1MT are weak, while its administration potentiates the activity of several chemotherapeutic drugs such as cyclophosphamide or paclitaxel (Muller el at 2005).

Selective TDO2 inhibition by LM-10 (trans-6-Fluoro-3-[2-(1H-tetrazol-5-yl)vinyl]-1H-indole) was recently presented to possess benefits in experimental model of mastocytoma. LM-10 does not inhibit IDO and has a higher solubility and bioavailability when compared to 1MT (Pilotte et al 2012).

IDO1 is overexpressed in many types of tumour with the highest frequency found in colorectal and pancreas cancers and glioblastoma (Uyttenhove et al 2003). Moreover, increasing expression of IDO1 in colorectal cancer is associated with a poor prognosis (Ferdinande et al 2012). It was shown recently that TDO2 is a new enzyme responsible of tryptophan catabolism in major types of cancer (Pilotte et al 2012).

Furthermore, TDO2 overexpression was presented to promote glioblastoma progression by acting on both the immune and the tumoral compartments (Opitz et al 2012). In Table 1 we summarize the different expression level of IDO1 and TDO2 in different types of tumors with their respective clinical significance.

Several teams aim to define new pharmacological inhibitor of IDO1 and TDO2 with better specificity and higher affinity than 1MT. Several natural compounds have been described as good candidates to limit IDO1 activity. However, this approach has various limitations.

Indeed, IDO1 inhibition cannot block the kynurenine pathway completely since TDO2 has the same catalyzing effect. In the same way, TDO2 inhibition would not be enough to block the kynurenine pathway. A combination of TDO2 and IDO1 inhibitors (Pilotte et al, 2012), or the identification of compounds able to block both IDO1 and TDO2 has been considered.

However, the inhibition of both IDO1 and TDO2 could have unprecedented side effects, e.g. by completely blocking out an entire metabolic pathway.

### SUMMARY OF THE INVENTION

It is an object of the present invention to overcome the limitations related to therapies addressing IDO1 and/or TDO2 as a therapeutic target for cancer treatment.

It is another object of the present invention to provide new therapeutic options for cancer.

It is another object of the present invention to provide a treatment against a neoplastic disease, which has good efficacy and/or little side effects.

It is yet another object of the present invention to provide tools for diagnosis, prognosis, risk assessment and/or prediction of a neoplastic disease is provided.

These objects are achieved by the subject matter of the independent claims, while the dependent claims as well as the specification disclose further preferred embodiments.

### DETAILED DESCRIPTION OF THE FIGURES

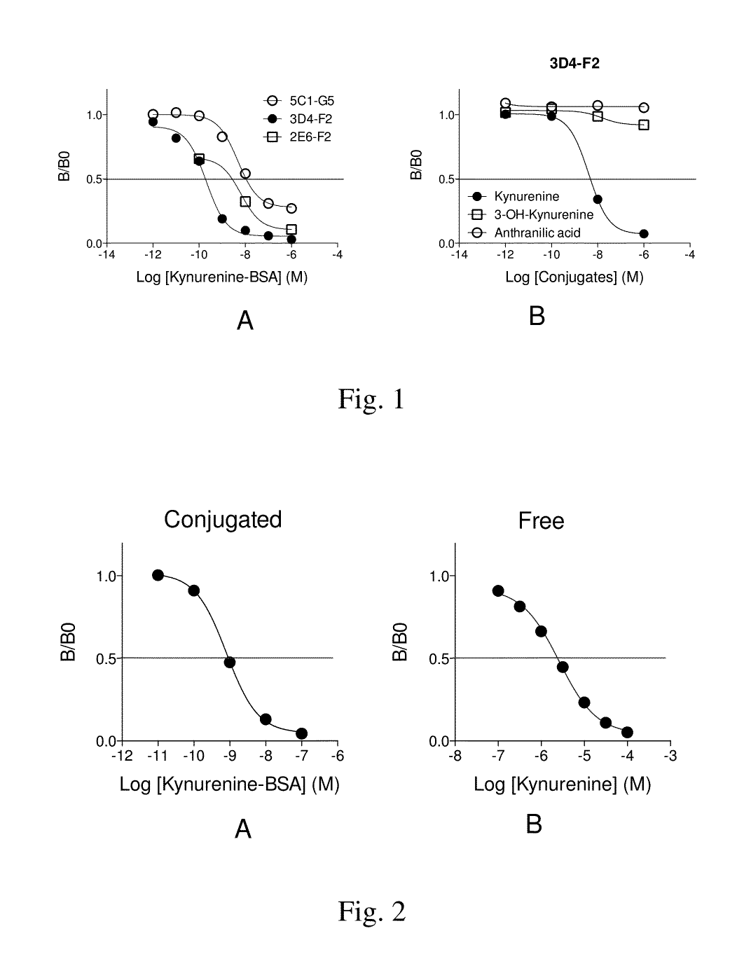

FIG. 1: Immunochemical characterization of monoclonal antibodies directed against carrier conjugated L-Kynurenine.

A) Affinity comparison of the different monoclonal antibodies was performed with an ELISA competition assay and shows a higher affinity for the 3D4-F2.

B) Specificity of the 3D4-F2 antibody was evaluated with an ELISA competition assay and shown no cross-reaction with either the anthranilic or 3-hydroxykynurenine conjugates while the kynurenine conjugate was recognized at an affinity around 10⁻⁹M

FIG. 2: Detection of both conjugated and free Kynurenine in a biological fluid using EIA (Enzyme immunoassay).

A) An increasing concentration of Kynurenine-BSA conjugate was incubated with 3D4-F2 mAb (at 0.01 mg/ml) for 1 hour at 37° C. The HRP-Kynurenine conjugate (Tracer) at 1 μg/mL was added and the solution was incubated on a maxisporp plate coated with anti mouse IgG. Reaction was revealed using TetraMethylBenzidine (TMB). The results are expressed as B/B0, where B0 is the OD (Optical density) value obtained with the antibodies (3D4-F2) and the tracer alone while B is the OD value obtained in the presence of a specific concentration of Kynurenine-BSA conjugate.

B) An increasing concentration of free Kynurenine derivatized on proteins by mean of carbodiimide crosslinkage was incubated with 3D4-F2 mAb (at 0.01 mg/ml) for 1 hour at 37° C. The HRP-Kynurenine conjugate (Tracer) at 1 μg/mL was added and the solution was incubated on a maxisporp plate coated with anti mouse IgG. Reaction was revealed using TetraMethylBenzidine (TMB). The results are expressed as B/B0, where B0 is the OD (Optical density) value obtained with the antibodies (3D4-F2) and the tracer alone while B is the OD value obtained in the presence of a specific concentration of free kynurenine derivatized on proteins.

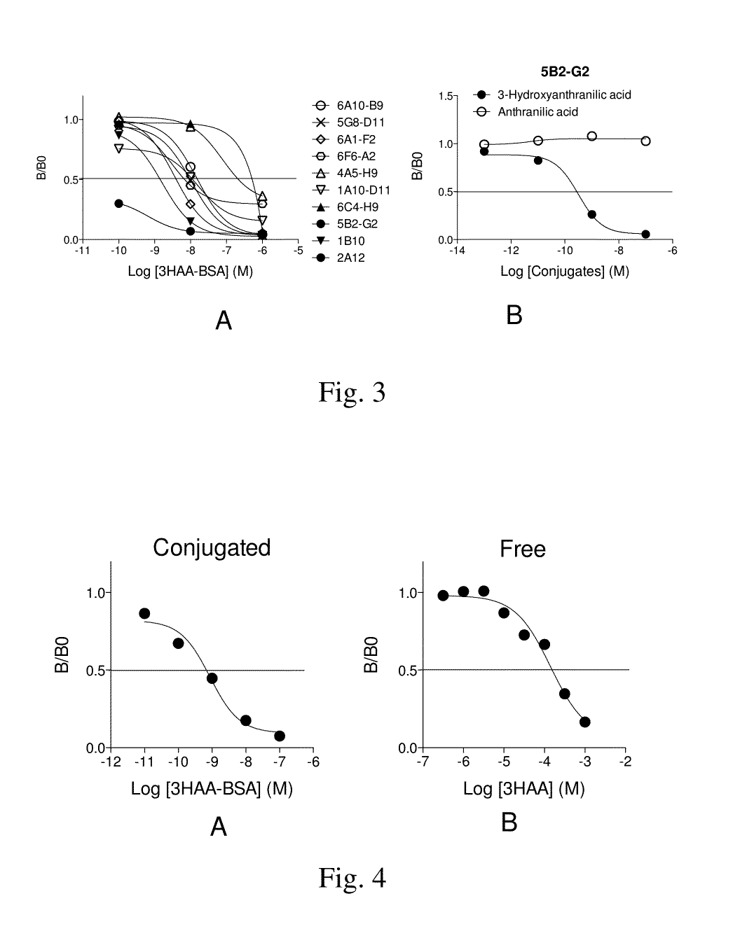

FIG. 3: Immunochemical characterization of monoclonal antibodies directed against carrier conjugated 3HAA.

A) Affinity comparison of the different monoclonal antibodies was performed with an ELISA competition assay and shows a higher affinity for the 5B2-G2 and 6F6-A2.

B) Specificity of the 5B2-G2 antibody was evaluated with a competition assay and shown no cross-reaction with the anthranilic conjugate while 3HAA conjugate was recognized at an affinity around 10⁻¹⁰M.

FIG. 4: Detection of both conjugated and free 3HAA in a biological fluid using EIA.

A) An increasing concentration of 3HAA-BSA conjugate was incubated with 5B2-G2 mAb (at 0.01 mg/ml) for 1 hour at 37° C. The HRP-3HAA conjugate (Tracer) at 1 μg/mL was added and the solution was incubated on a maxisporp plate coated with anti mouse IgG. Reaction was revealed using TetraMethylBenzidine (TMB). The results are expressed as B/B0, where B0 is the OD (Optical density) value obtained with only the antibodies (5B2-G2) and the tracer while B is the OD value obtained in the presence of a specific concentration of 3HAA-BSA conjugate.

B) An increasing concentration of free 3HAA derivatized on proteins by mean of ethylchloroformate crosslinkage was incubated with 5B2-G2 mAb (at 0.01 mg/ml) for 1 hour at 37° C. The HRP-3HAA conjugate (Tracer) at 1 μg/mL was added and the solution was incubated on a maxisporp plate coated with anti mouse IgG. Reaction was revealed using TetraMethylBenzidine (TMB). The results are expressed as B/B0, where B0 is the OD (Optical density) value obtained with only the antibodies (5B2-G2) and the tracer while B is the OD value obtained in the presence of a specific concentration of free 3HAA derivatized on proteins.

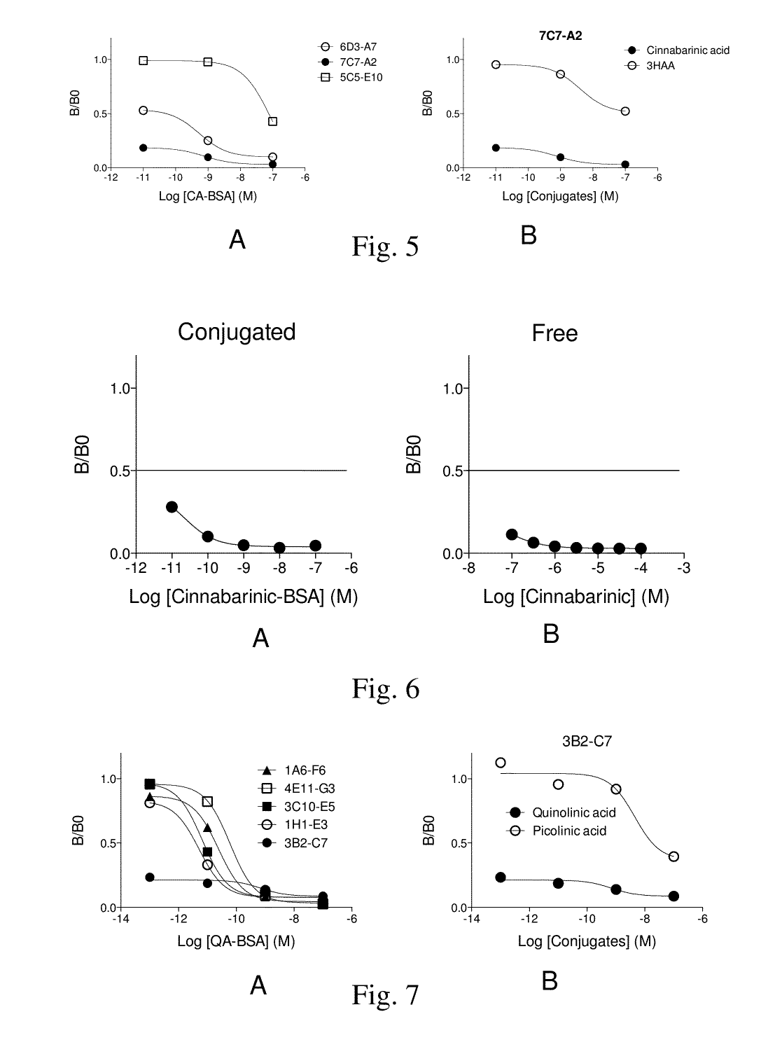

FIG. 5: Immunochemical characterization of monoclonal antibodies directed against carrier conjugated Cinnabarinic Acid.

A) Affinity comparison of the different monoclonal antibodies was performed with an ELISA competition assay and shows a higher affinity for the 7C7-A2.

B) Specificity of the 7C7-A2 antibody was evaluated with a competition assay and shown a weak cross reaction with the 3-hydroxykanthranilic acid (3HAA) conjugate while the kynurenine conjugate was recognized at an affinity <10⁻¹¹M.

FIG. 6: Detection of both conjugated and free Cinnabarinic Acid in a biological fluid using EIA.

A) An increasing concentration of CA-BSA conjugate was incubated with 7C7-A2 mAb (cell culture supernatant diluted 10 times) for 1 hour at 37° C. The HRP-CA conjugate (Tracer) at 0.03 μg/mL was added and the solution was incubated on a maxisporp plate coated with anti mouse IgG. Reaction was revealed using TetraMethylBenzidine (TMB). The results are expressed as B/B0, where B0 is the OD (Optical density) value obtained with the antibodies (7C7-A2) and the tracer alone while B is the OD value obtained in the presence of a specific concentration of CA-BSA conjugate.

B) An increasing concentration of free CA derivatized on proteins by mean of carbodiimide crosslinkage was incubated with 7C7-A2 mAb (cell culture supernatant diluted 10 times) for 1 hour at 37° C. The HRP-CA conjugate (Tracer) at 0.03 μg/mL was added and the solution was incubated on a maxisporp plate coated with anti mouse IgG. Reaction was revealed using TetraMethylBenzidine (TMB). The results are expressed as B/B0, where B0 is the OD (Optical density) value obtained with the antibodies (7C7-A2) and the tracer alone while B is the OD value obtained in the presence of a specific concentration of free kynurenine derivatized on proteins.

FIG. 7: Immunochemical characterization of monoclonal antibodies directed against carrier conjugated Quinolinic acid.

A) Affinity comparison of the different monoclonal antibodies was performed with an ELISA competition assay and shows a higher affinity for the 3B2-C7.

B) Specificity of the 3B2-C7 antibody was evaluated with a competition assay and shown a weak cross reaction with the Picolinic acid (3HAA) conjugate while the Quinolinic conjugate was recognized at an affinity <10⁻¹³M.

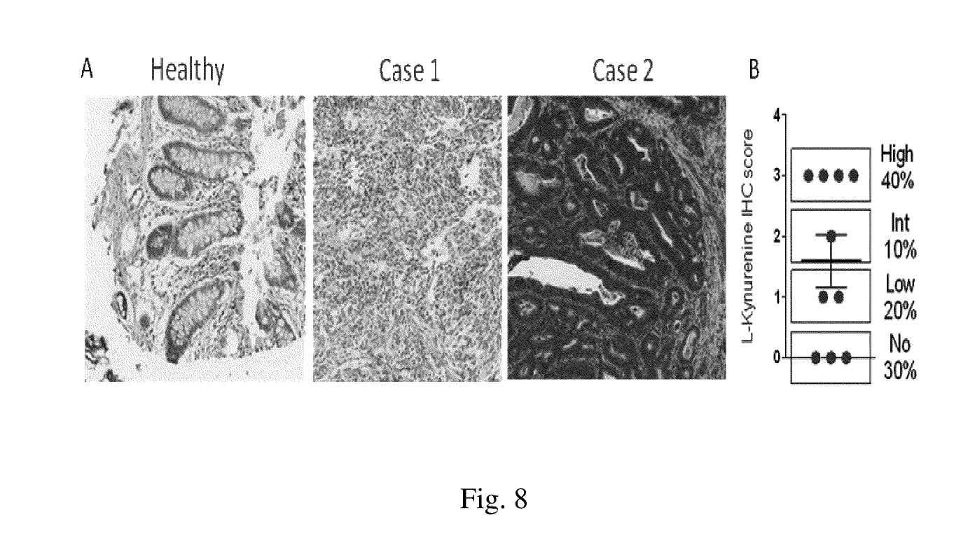

FIG. 8: L-Kynurenine detection in human colon carcinomas

A) L-Kynurenine presence was evaluated by IHC in 10 colon carcinomas and 2 normal colons on a tissue micro array section obtained from US Biomax (USA). A weak staining was observed in a healthy colon (Healthy) while Case 1 shows absence of L-kynurenine and Case 2 shows a strong L-Kynurenine production/accumulation in the cytoplasm of tumour cells.

B) Graphical representation of the staining heterogeneity among colon cancer patients. 40% of patients show a strong staining, 10% of patients an intermediate staining, 20% of patients a weak staining and 30% of patients an absence of staining. These data reveal that L-Kynurenine production pattern revealed by IHC can stratify patients harboring colon tumours.

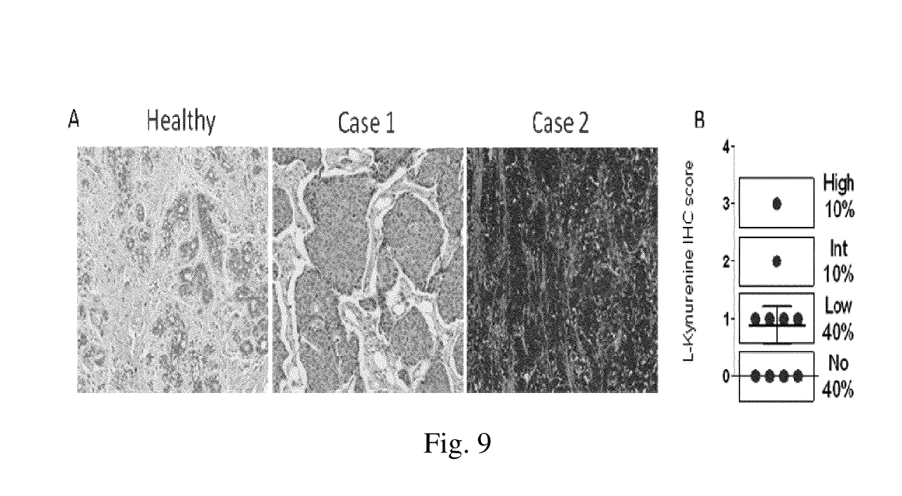

FIG. 9: L-Kynurenine detection in human breast carcinomas

A) L-Kynurenine presence was evaluated by IHC in 10 breast carcinomas and 2 normal colons on a tissue micro array section obtained from US Biomax (USA). A weak staining was observed in a healthy breast epithelium (Healthy) while Case 1 shows absence of L-kynurenine and Case 2 shows a strong L-Kynurenine production/accumulation in the cytoplasm of tumour cells and cells from the tumoural stroma.

B) Graphical representation of the staining heterogeneity among breast cancer patients. 10% of patients show a strong staining, 10% of patients an intermediate staining, 40% of patients a weak staining and 40% of patients an absence of staining. These data reveal that L-Kynurenine production pattern, revealed by IHC, can stratify patients harboring breast carcinomas.

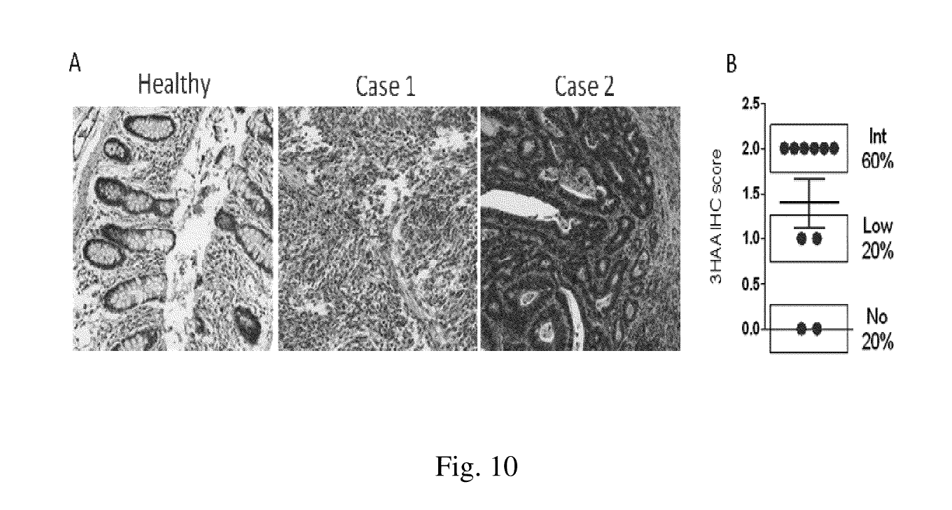

FIG. 10: 3HAA detection in human colon carcinomas

A) 3HAA presence was evaluated by IHC in 10 colon carcinomas and 2 normal colons on a tissue micro array section obtained from US Biomax (USA). An intermediate staining was observed in a healthy colon (Healthy) while Case 1 shows absence of 3HAA and Case 2 shows an intermediate 3HAA production/accumulation in the cytoplasm of tumour cells.

B) Graphical representation of the staining heterogeneity among colon cancer patients. 60% of patients show an intermediate staining, 20% of patients low staining and 20% of patients an absence of staining. These data reveal that 3HAA production pattern, revealed by IHC, can stratify patients harboring colon tumours.

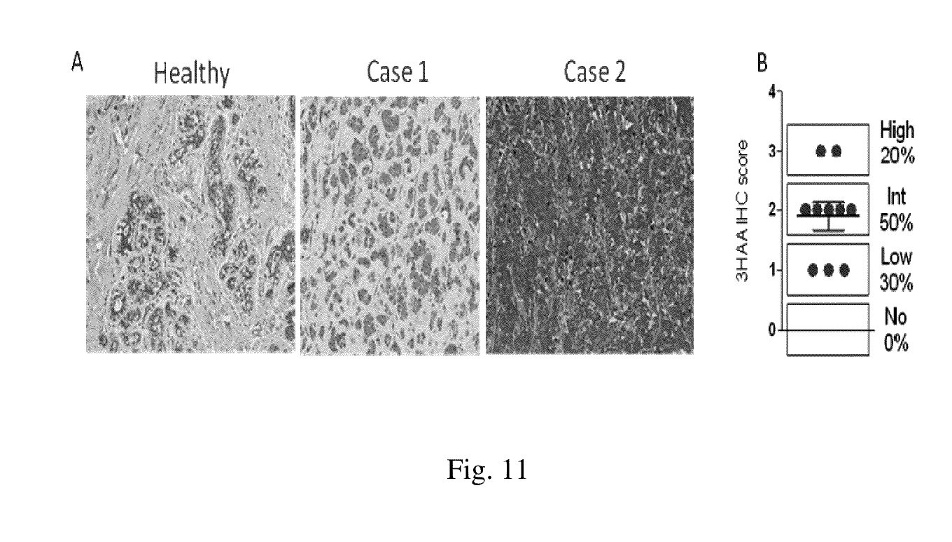

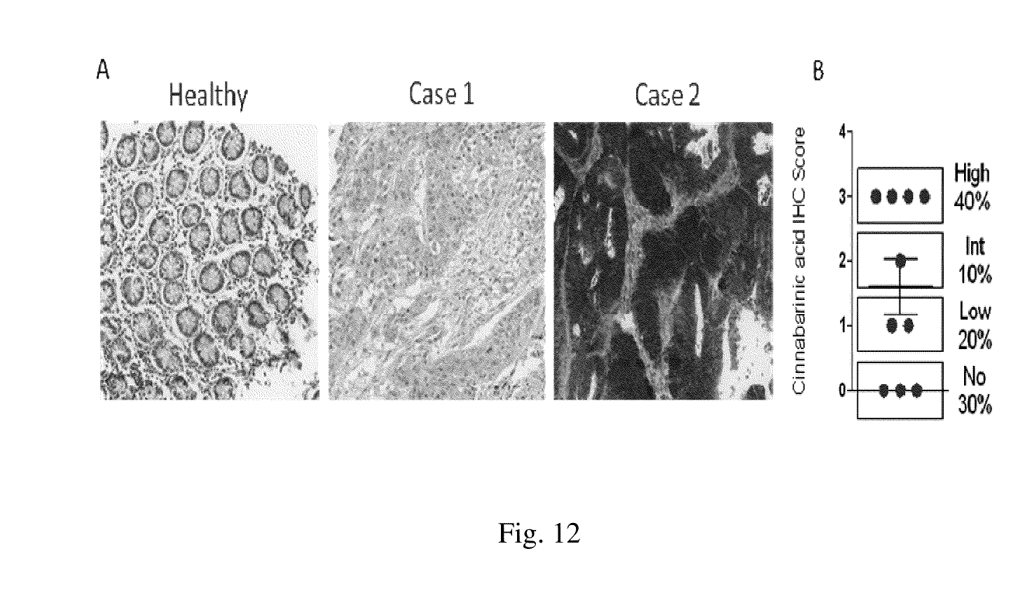

FIG. 11: 3HAA detection in human breast carcinomas

A) 3HAA presence was evaluated by IHC in 10 breast carcinomas and 2 normal colons on a tissue micro array section obtained from US Biomax (USA). An intermediate staining was observed in a healthy breast epithelium (Healthy) while Case 1 shows weak production of 3HAA and Case 2 shows a strong 3HAA production/accumulation in the cytoplasm of tumour cells and cells from the tumoural stroma.

B) Graphical representation of the staining heterogeneity among breast cancer patients. 20% of patients shows a strong staining, 50% of patients an intermediate staining, 30% of patients a weak staining. These data reveal that 3HAA production pattern, revealed by IHC, can stratify patients harbouring breast carcinomas.

FIG. 12: Cinnabarinic detection in human colon carcinomas

A) Cinnabarinic Acid (CA) presence was evaluated by IHC in 10 colon carcinomas and 2 normal colons on a tissue micro array section obtained from US Biomax (USA). No staining was observed in a healthy colon (Healthy) while Case 1 shows a weak production of CA and Case 2 shows a strong CA production/accumulation in the cytoplasm of tumour cells.

B) Graphical representation of the staining heterogeneity among colon cancer patients. 40% of patients show a strong staining, 10% of patients an intermediate staining, 20% of patients a weak staining and 30% of patients an absence of staining. These data reveal that CA production pattern, revealed by IHC, can stratify patients harbouring colon tumours.

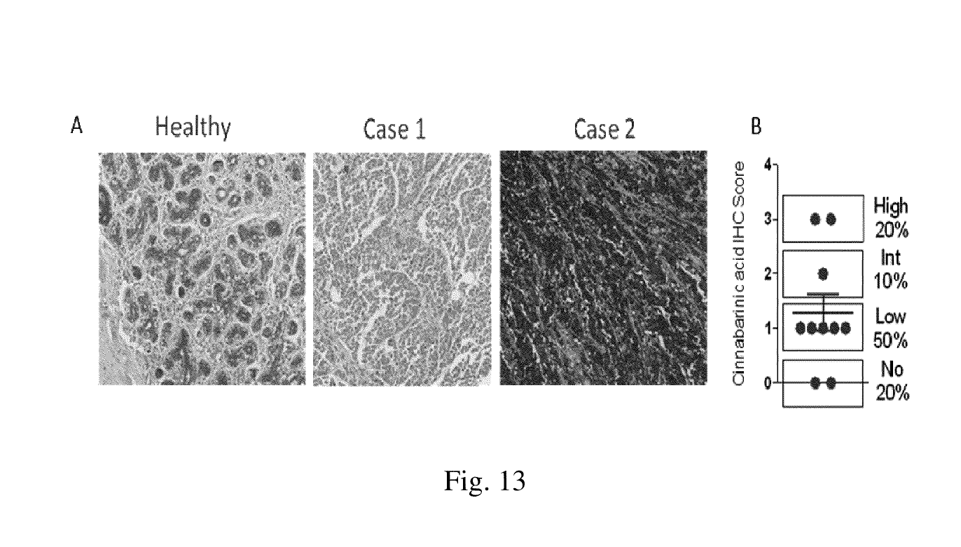

FIG. 13: Cinnabarinic Acid detection in human breast carcinomas

A) CA presence was evaluated by IHC in 10 breast carcinomas and 2 normal colons on a tissue micro array section obtained from US Biomax (USA). An intermediate staining was observed in a healthy breast epithelium (Healthy) while Case 1 shows absence of CA and Case 2 shows a strong CA production/accumulation in the cytoplasm of tumour cells and cells from the tumoral stroma.

B) Graphical representation of the staining heterogeneity among breast cancer patients. 20% of patients show a strong staining, 10% of patients an intermediate staining, 50% of patients a weak staining and 20% of patients an absence of staining. These data reveal that CA production pattern, revealed by IHC, can stratify patients harboring breast carcinomas.

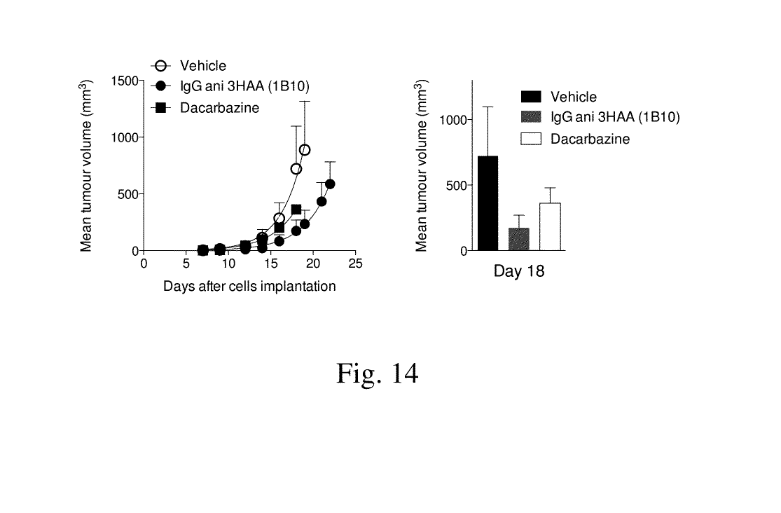

FIG. 14: Benefits of 3HAA monoclonal antibodies in a mouse model of melanoma induced by B16-F10 tumour cells implantation into C57BL/6 mice.

A) Tumour size was followed for 22 days after treatment with either vehicle, IgG anti 3HAA (1B10) or Dacarbazine. 100 μg of IgG anti 3HAA were administered subcutaneously at day 6 (at a time when tumours were detectable), 13 and 20. Dacarbazine was administered intra-peritoneally at 80 mg/kg. The treatment started at day 6 and was repeated once every 2 weeks for 4 consecutive times; chemotherapy cycles were repeated every 4 days. These results show a substantial benefit of the IgG anti 3HAA with higher efficacy when compared to dacarbazine.

B) Graphical representation of the tumour size at Day 18 after B16-F10 inoculation shows the same result as in A.

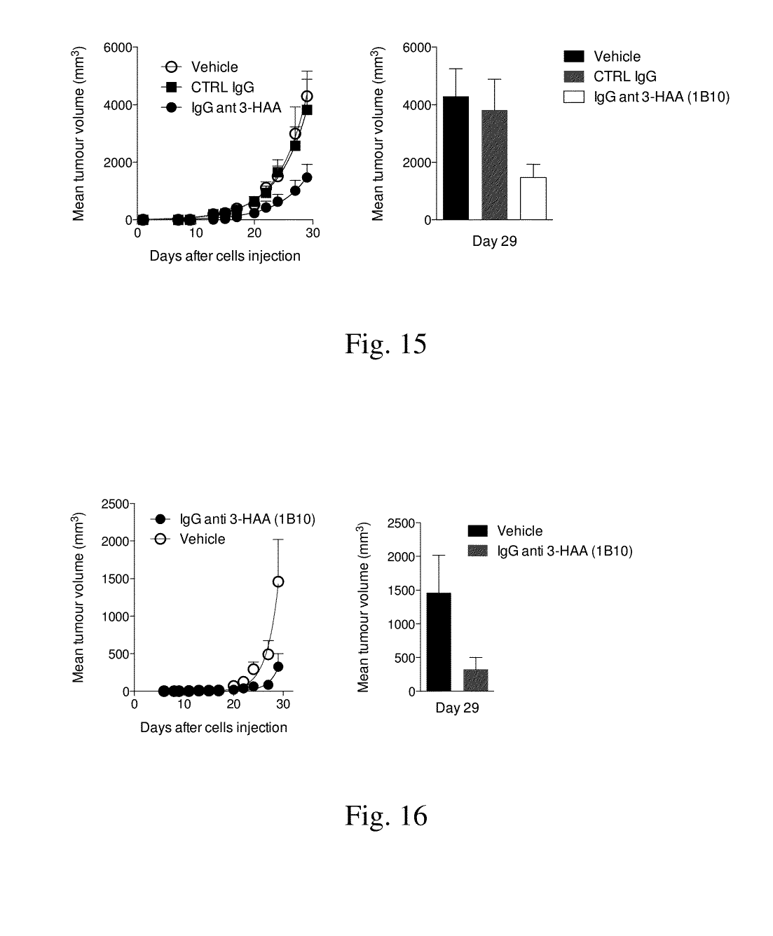

FIG. 15: Benefits of 3HAA monoclonal antibodies in a mouse model of Glioblastoma induced by GL261 tumour cells implantation into C57BL/6 mice.

A) Tumour size was followed for 29 days after treatment with either vehicle, IgG anti 3HAA (1B10) or control IgG. 50 μg of IgG anti 3HAA were administered subcutaneously 1 day before cells implantation and repeated once a week. These results show a benefit of the IgG anti 3HAA when administered as preventive treatment.

B) Graphical representation of the tumour size at Day 29 after GL261 inoculation shows the same result as in A.

FIG. 16: Benefits of 3HAA monoclonal antibodies in a mouse model of Glioblastoma induced by GL261 tumour cells implantation into C57BL/6 mice.

A) Tumour size was followed for 29 days after treatment with either vehicle, IgG anti 3HAA (1B10) or control IgG. 50 μg of IgG anti 3HAA were administered subcutaneously at day 6 (at a time when tumours were detectable), 13, 20 and 27. These results show a benefit of the IgG anti 3HAA.

B) Graphical representation of the tumour size at Day 29 after GL261 inoculation shows the same result as in A.

FIG. 17: Benefits of 3HAA monoclonal antibodies in an intracerebral model of glioblastoma obtained by GL261 tumour cells implantation into C57BL/6 mice.

A) Detection of 3HAA in tumour sections obtained from mice implanted intracerebrally with GL261. 3HAA was both detected in surrounding reactive astrocytes and tumour cells with a likely polarized localization.

B) Mice were treated 6 days after intracerebral GL261 implantation with 100 μg of IgG anti 3HAA (1B10) injected subcutaneously and was repeated once a weak. 29 Days after cell implantation, brains were taken, systematically sliced and tumour area photographed and measured. When compared to vehicle treated mice, IgG anti 3HAA reduced significantly the tumour volume.

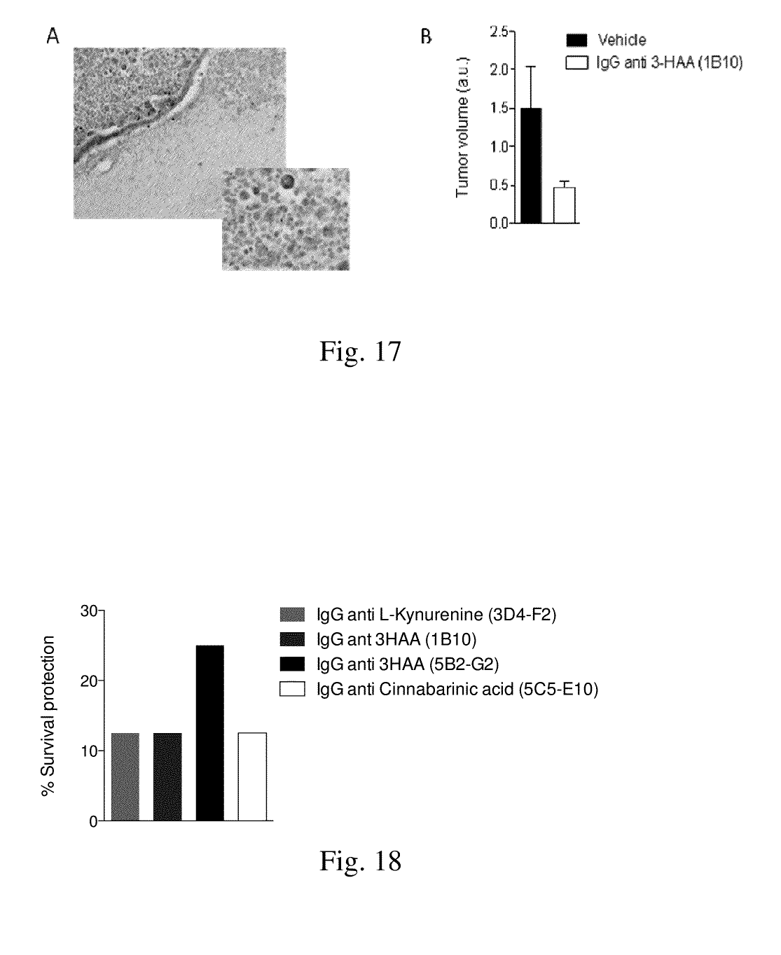

FIG. 18: Benefits of monoclonal antibodies directed against either L-Kynurenine (3D4-F2), 3HAA (1B10 and 5B2-G2) and Cinnabarinic Acid (5C5-E10) in an intracerebral model of Glioblastoma induced by GL261 tumour cells implantation into C57BL/6 mice.

30 Days after tumour cells implantation, 0% of mice treated with the vehicle survived while the % of survival protection was 12.5% for IgG anti 3HAA (1B10), 25% for IgG anti 3HAA (5B2-G2), 12.5% for IgG anti L-Kynurenine (3D4-F2) and 12.5% for IgG anti Cinnabarinic Acid (5C5-E10). These results show a benefit of targeting kynurenine pathway metabolites, and particularly L-Kynurenine, 3HAA and Cinnabarinic Acid, in the treatment of tumors.

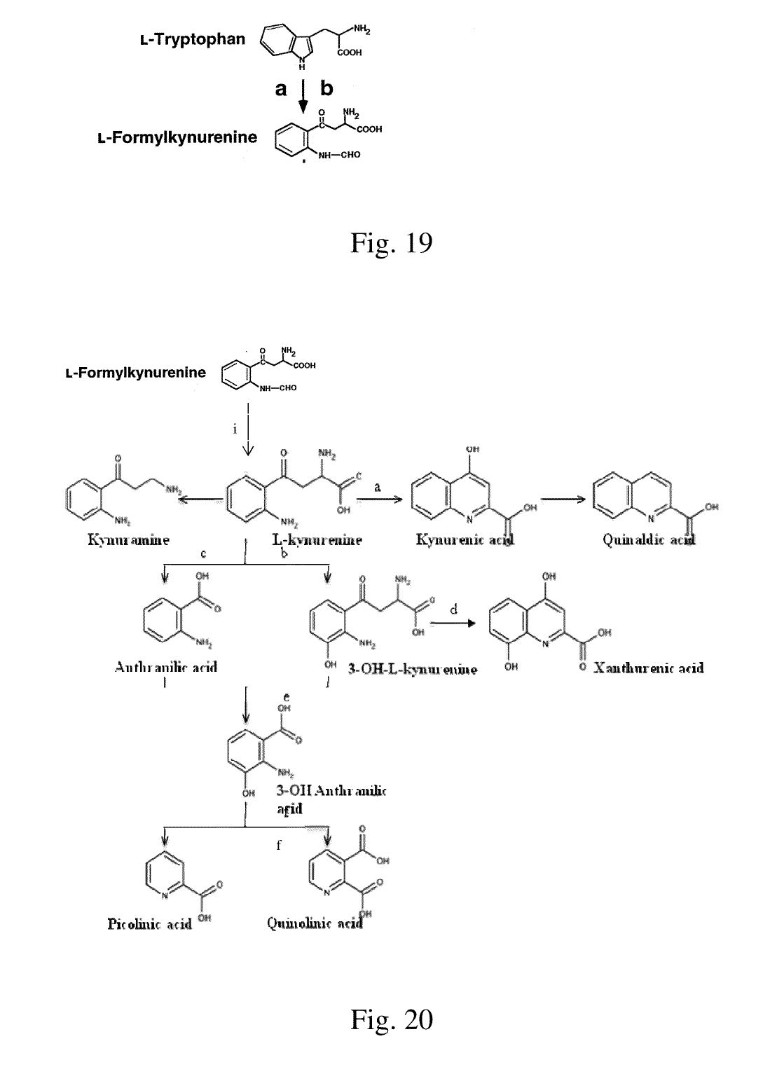

FIG. 19: Entry reaction which initiates the kynurenine pathway (L-Tryoptophan->L-Formylkynurenine, but is not part thereof. The step is catalyzed by either a) Indoleamine 2,3-dioxygenase (IDO1) or b) Tryptophan 2,3-dioxygenase (TDO2). If one of the two is blocked, the reaction can still take place, while blocking both may have severe side effects.

FIG. 20: Overview of the kynurenine pathway with its enzymes and metabolites. The enzymes are as follows: i) Kynurenine formamidase, a) Kynurenine amino-transferase, b) Kynurenine 3-hydroxylase (also called Kynurenine mono-oxygenase), c) Kynureninase (also called L-Kynurenine hydrolase), d) Kynurenine amino-transferase, e) Kynureninase (also called L-Kynurenine hydrolase), and f) 3-Hydroxyanthranilic Acid oxygenase (also called 3-Hydroxanthranilate dioxygenase).

The metabolites are as follows: L-Formylkynurenine, Kynuramine, L-Kynurenine, Kynurenic Acid, 3-hydroxyL-kynurenine, Anthranilic Acid, 3-hydroxyanthranilic Acid, Xanthurenic Acid, Quinaldic Acid, Picolinioc Acid and/or Quinolinic Acid

Please note that some metabolites and enzymes of the kynurenine pathway are not shown.

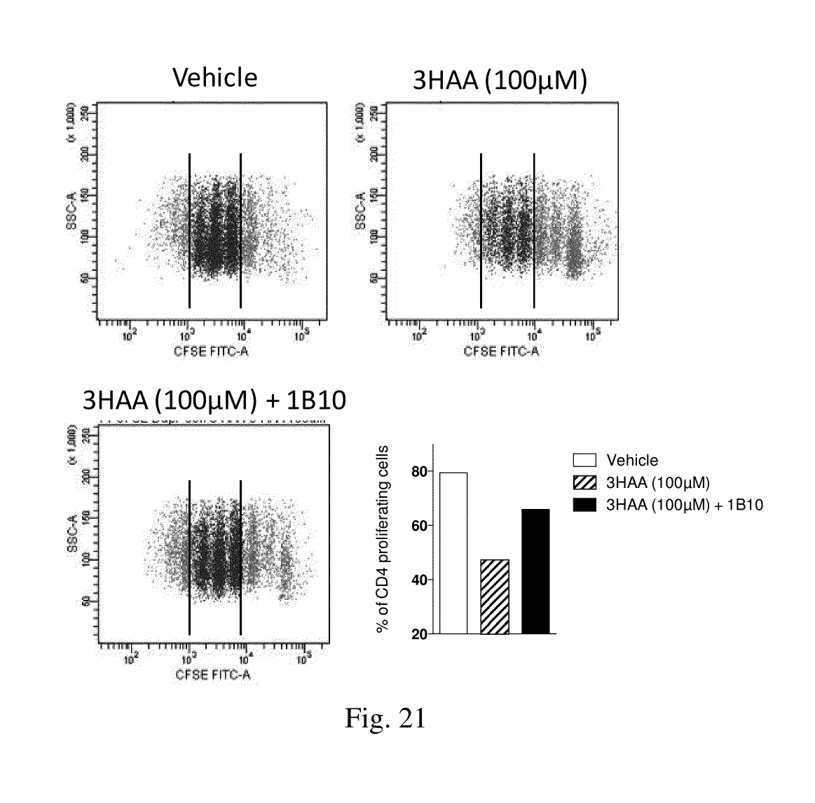

FIG. 21: Suppression of anti-proliferative properties of 3HAA on helper T cells by a 3HAA monoclonal antibody (1B10). CD4 positive T cells were activated by anti CD3/CD28 antibodies cocktail and incubated for 96 hours with either vehicle, 3HAA at 100 μM or 3HAA (100 μM)+IgG anti 3HAA. As expected, anergy of T cells was induced by 3HAA, a phenomenon largely ablated when co-incubated with the 3HAA monoclonal antibody.

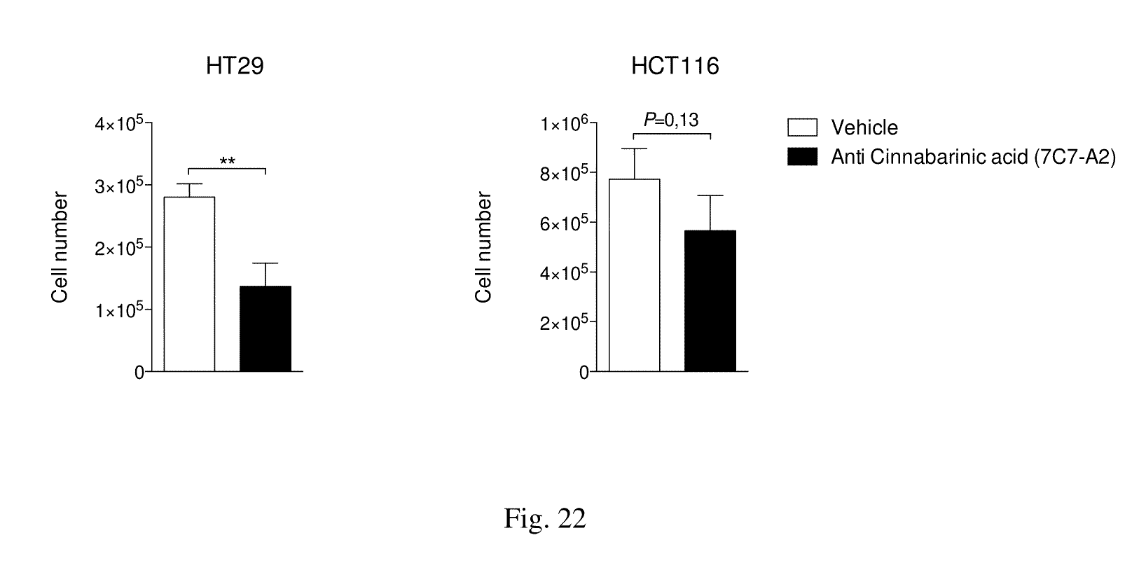

FIG. 22: Anti-proliferative effect of IgG anti Cinnabarinic acid on human colon cancer cell lines. HT29 and HCT116 cells were plated for 24 hours and incubated with 7C7-A2 culture supernatant for 48 hours. Cells were then counted and show a significant decrease of proliferation rate in HT29 while only a trend in the same way was observed in HCT116.

### DETAILED DESCRIPTION OF THE INVENTION

In one embodiment of the invention, a modulator of an enzyme and/or a metabolite of the kynurenine pathway is provided.

As used herein, the term “modulator of an enzyme and/or a metabolite of the kynurenine pathway” refers to a substance that affects at least one selected from the group consisting of the formation, concentration, availability, metabolism and/or effect of an enzyme and/or a metabolite of the kynurenine pathway.

Such modulator does not necessarily have to bind to said enzyme and/or a metabolite of the kynurenine pathway. It is sufficient if said modulator exerts its effect even without binding, e.g., indirectly, e.g. by (i) affecting an enzyme which is causative for the formation of said target metabolite, (ii) affecting a co-factor which the target enzyme needs, and/or (iii) affecting the molecular target of said enzyme and/or a metabolite, in such way limiting a potential pathological effect of the latter.

Said enzyme and/or a metabolite of the kynurenine pathway can either be in a soluble form, or attached to another moiety (e.g., bound to a membrane or the like, attached to a cofactor, or the like).

As used herein, the term “kynurenine pathway” encompasses enzymes and metabolites of said pathway with the exception of indoleamine 2,3 dioxygenase 1 and 2 (IDO 1/2) and tryptophan 2,3 dioxygenase 2 (TDO2). Either of the former two enzymes catalyzes the formation of N-Formylkynurenine (NFK) from Tryptophan.

An overview of the kynurenine pathway with its enzymes and metabolites is shown in FIG. 20

The kynurenine pathway is a metabolic pathway leading to the production of nicotinamide adenine dinucleotide (NAD+) from the degradation of the essential amino acid tryptophan. The kynurenine pathway is involved in physiological functions such as behavior, sleep, thermo-regulation and pregnancy.

There is evidence of kynurenine pathway involvement in neurotoxic mechanisms associated with several inflammatory neurological diseases. Although the pathway is activated in these disorders, kynurenine and its metabolites can play both neurotoxic and neuroprotective roles by influencing neurotransmitter functions and inflammatory pathways peripherally and within the central nervous system.

The finding that the inhibition of an enzyme and/or a metabolite of the kynurenine pathway can provide a feasible option to treat a human or animal patient came quite surprisingly, particularly in view of the fact that it is known that anti-IDO1 or anti-TDO2 treatments have limited efficacy while blocking both could result in significant side effects.

In a preferred embodiment of the present invention, said modulator is an antagonist to said enzyme and/or a metabolite of the kynurenine pathway.

As used herein, the term “antagonist” shall encompass all moieties that have an affinity to an enzyme and/or a metabolite of the kynurenine pathway, or a conjugate thereof, but no efficacy. This means, for example, that (i) upon binding of said antagonist to the enzyme and/or metabolite no physiological function is elicited, or a dampened or altered physiological function is elicited, (ii) binding of said antagonist to the enzyme and/or metabolite inhibits the binding thereof to its physiological counterpart, or (iii) binding of said antagonist to said metabolite inhibits metabolism thereof

Binding will thus disrupt the interaction between the enzyme or metabolite, or a conjugate thereof to its physiological target, and thus inhibit its role in pathological processes.

In a preferred embodiment it is provided that administration of said modulator and/or antagonist to a human or animal body affects at least one selected from the group consisting of the formation, concentration, availability and/or effect of 3HAA (3-Hydroxy Anthranilic Acid), L-Kynurenine, Quinolinic Acid and/or Cinnabarinic Acid.

As used herein, the term “formation of a metabolite” means metabolic synthesis thereof, e.g., in the kynurenine pathway. As used herein, the term “concentration of a metabolite” means concentration thereof in one or more selected tissues, plasma serum levels, and the like. As used herein, the term “effect of a metabolite” relates to any downstream function the metabolite may have. By a non-restricting example, such function may be, e.g., a metabolite function as well as a co-factor function or a 2^{nd} messenger function.

The inventors have shown that 3HAA, L-Kynurenine, Quinolinic Acid and/or Cinnabarinic Acid seem to be key metabolites that may be used as a therapeutic target. Without being bound to theory, it is assumed that, for example, 3HAA, L-Kynurenine, Quinolinic Acid and/or Cinnabarinic Acid seem to play a key role in the immune escape and tumor growth, blocking of which may thus restore the function of the immune system against the tumor and its oncological properties.

Also, the finding that blocking of 3HAA may have a desirable effect is surprising. The prior art, particularly the prior art which discusses the use of IDO1 inhibitors and TDO2 inhibitors, provides no input with respect to which metabolite or enzyme downstream thereof is the cancer-promoting agent which may be blocked by inhibition of IDO1 and/or TDO2. Same findings are deemed applicable to other metabolites of the Kynurenine pathway, like L-Kynurenine, Quinolinic Acid and/or Cinnabarinic Acid.

In another preferred embodiment, said enzyme of the kynurenine pathway is at least one selected from the group consisting of Kynurenine formamidase, Kynurenine amino-transferase, Kynurenine 3-hydroxylase (also called Kynurenine mono-oxygenase), Kynureninase (also called L-Kynurenine hydrolase), Kynurenine amino-transferase, and/or 3-Hydroxyanthranilic Acid oxygenase (also called 3-Hydroxanthranilate dioxygenase).

In another preferred embodiment, said metabolite of the kynurenine pathway is at least one selected from the group consisting of N-Formylkynurenine, D and/or L-Kynurenine, Kynurenic acid, Quinaldic acid, Kynuramine, 3-hydroxy-L-kynurenine, 3-hydroxy-D-kynurenine, Xanthommatin, Anthranilic Acid, Xanthurenic Acid, 3-Hydroxy Anthranilic Acid, Picolinioc Acid and/or Quinolinic Acid and/or Cinnabarinic Acid.

An overview of particularly preferred enzymes and metabolites of the kynurenine pathway is shown in FIG. 20.

In a particularly preferred embodiment, the modulator and/or antagonist to an enzyme and/or a metabolite of the kynurenine pathway is an antagonist to 3HAA (3-Hydroxy Anthranilic Acid), L-Kynurenine, Quinolinic Acid and/or Cinnabarinic Acid. Again, the respective metabolite can be present either be in a soluble form, or attached to another moiety (e.g., bound to a membrane or the like, attached to a cofactor, or the like). Without being bound to theory, such antagonist can inactivate the cancer-promoting effect of the metabolite, e.g., by avoiding binding thereof to an enzyme or receptor, or avoiding further metabolic degradation thereof

In another preferred embodiment, the modulator and/or antagonist to an enzyme and/or a metabolite of the kynurenine pathway is an antagonist to the enzyme Kynureninase. This enzyme catalyzes one step of the kynurenine pathway, namely the transformation of 3-Hydroxykynurenine to 3HAA (3-Hydroxy Anthranilic Acid). Blocking of this enzyme can effectively reduce the formation and/or concentration of 3HAA (3-Hydroxy Anthranilic Acid). Two non-restricting examples of preferred antagonists to Kynureninase are O-methoxybenzoylalanine 2-amino-4-[3′-hydroxyphenyl]-4-hydroxybutanoic acid.

In another preferred embodiment, said human or animal suffers from a neoplastic disease. The term “neoplastic disease”, as used herein, refers to an abnormal state or condition of cells or tissue characterized by rapidly proliferating cell growth or neoplasm. In a more specific meaning, the term relates to cancerous processes, e.g., tumors and/or leukemias.

In another preferred embodiment, said human or animal suffers from a disease caused by over- or underabundancy of an enzyme and/or metabolite of the kynurenine pathway.

Preferably, said neoplastic disease is at least one disease characterized by tryptophan metabolism exaggeration along the kynurenine pathway. Tryptophan metabolism exaggeration is defined as a decrease in tryptophan level and/or increase of one of the kynurenine pathway metabolites.

More preferably said neoplastic disease is at least one disease characterized by any form of activity of Indoleamine 2,3-dioxygenase 1 (IDO1) and/or Indoleamine 2,3-dioxygenase 2 (IDO2) and/or Tryptophan 2,3-dioxygenase (TDO2).

Table 1 shows a non-exhaustive list of cancers characterized by overexpression of IDO1 and/or TDO2. Therein, “IHC” means “Immunohistochemistry” and “Tregs” mean “Regulatory T cells”, “↓L” means “reduces” and “↑” means “increases”.

TABLE 1
cancers characterized by overexpression of IDO1 and/or TDO2

| | IDO1/ | | Prognosis | |
|---|---|---|---|---|
| Types of cancer | TDO2 | Technique | significance | Ref. |
| Cutaneous basocellular | IDO | IHC | ND | 16 |
| carcinomas | | | | |
| Cervical carcinomas | IDO | IHC | ? | 23 |
| | IDO | IHC | ND | 16 |
| | TDO | IHC | ND | 19 |
| Colorectal carcinomas | IDO | IHC | ↓survival | 17 |
| | IDO | IHC | ↑métastasis | 24 |
| | IDO | IHC | ND | 16 |
| | TDO | IHC | ND | 19 |
| Endometrial carcinomas | IDO | IHC | ↓survival | 25 |
| | | | ↑métastasis | |
| | IDO | IHC | ND | 16 |
| Gastric carcinomas | IDO | IHC | ? | 16 |
| Glioblastomas | TDO | IHC | ↓survival | 19 |
| | IDO | IHC | ND | 16 |
| Hepatocarcinomas | IDO | IHC | ↓survival | 26 |
| | | | ↑métastasis | |
| | IDO | IHC | ND | 16 |
| Acute myeloid leukaemia | IDO | ICC | ↑Tregs | 27 |
| Lymphomas | IDO | IHC | ND | 16 |
| | TDO | IHC | ND | 19 |
| Melanomas | IDO | IHC | ↓survival | 28 |
| | IDO | IHC | ND | 16 |
| Mesotheliomas | IDO | IHC | ND | 16 |
| Nasopharynx | IDO | IHC | ? | 29 |
| Oesophageal carcinomas | IDO | IHC | ND | 16 |
| Ovarian carcinomas | IDO | IHC | ↓survival | 30 |
| | IDO | IHC | ND | 16 |
| | TDO | IHC | ND | 19 |
| Pancreatic carcinomas | IDO | IHC | ↑Tregs | 31 |
| Small-cell lung carcinomas | IDO | IHC | ND | 16 |
| Non small-cell lung | IDO | IHC | ↓survival | 32 |
| carcinomas | IDO | IHC | ND | 16 |
| Prostatic carcinomas | IDO | IHC | ND | 16 |
| Renal cell carcinomas | IDO | IHC | ND | 16 |
| Sarcomas | IDO | IHC | ND | 16 |
| Sarcomas (Ewing) | TDO | IHC | ND | 19 |
| Breast carcinomas | IDO | IHC | ↑metastasis | 33 |
| | IDO | IHC | ND | 16 |
| Head and neck carcinomas | IDO | IHC | ND | 16 |
| Thyroid carcinomas | IDO | IHC | ND | 16 |
| Bladder carcinomas | IDO | IHC | ND | 16 |
| | TDO | IHC | ND | 19 |

It is important to understand that according to the definition of the present invention, neither Indoleamine 2,3-dioxygenase 1 (IDO1) nor Tryptophan 2,3-dioxygenase (TDO2) are members of the kynurenine pathway.

The above list reflects prior knowledge, which does not anticipate the subject matter of the present invention. Despite the abundancy of prior art related to IDO1 and/or TDO2, the role of enzymes and metabolites of the kynurenine pathway has always been overlooked. It is the accomplishment of the inventors that they have, for the first time, investigated the role of the kynurenine pathway for cancer genesis.

The inventors have, surprisingly, conceived that a relationship between the kynurenine pathway and cancer genesis exists. This finding applies to all cancers characterized by overexpression of IDO1 and/or TDO2.

In a very preferred embodiment, said neoplastic disease is selected from the group consisting of
Colorectal cancer
Breast cancer
Melanoma, and/or
Glioma and other tumors of the central nervous system

In another preferred embodiment, said modulator and/or antagonist to an enzyme and/or a metabolite of the kynurenine pathway serves to inhibit processes, in the human or animal, related to immune escape and/or immunoediting.

Tumor escape is a key factor in cancer progression, which has been neglected for long time. When a cell becomes malignant by transformation, i.e., when it becomes different from the “self”, it should be recognized and eliminated by the immune system. Accordingly, transformed cells are continuously eliminated in our organism by a functional immune system. Unfortunately, a tumor cell is, in some cases, able to repress the immune response. This process is called the immune escape. All processes related to the immune escape are characterized as “immunoediting”.

Preferably, said modulator and/or antagonist to an enzyme and/or a metabolite of the kynurenine pathway is at least one selected from the group consisting of
a monoclonal antibody (murine, chimeric, humanized, human)
a fragment or derivative thereof (e.g., Fab, Fab2, scFv)
a new antibody format
a fusion peptide comprising at least one domain capable of binding an enzyme and/or a metabolite of the kynurenine pathway
a antibody mimetic,
an aptamer, and/or
a small molecule antagonist.

The above list encompasses different classes of protein therapeutics, plus aptamers and small molecules. Before the different preferred embodiments are described in detail, it is important to mention that, once the skilled person has learned that an enzyme and/or a metabolite of the kynurenine pathway is a promising target for a antagonistic therapy, the skilled person will be able to provide an antagonist from the above list without solely by using standard methods of creating a library and screening the latter.

As used herein, the term “monoclonal antibody (mAb)”, shall refer to an antibody composition having a homogenous antibody population, i.e., a homogeneous population consisting of a whole immunoglobulin, or a fragment or derivative thereof. Particularly preferred, such antibody is selected from the group consisting of IgG, IgD, IgE, IgA and/or IgM, or a fragment or derivative thereof.

As used herein, the term “fragment” shall refer to fragments of such antibody retaining, in some cases, target binding capacities, e.g.
a CDR (complementarity determining region)
a hypervariable region,
a variable domain (Fv)
an IgG heavy chain (consisting of VH, CH1 hinge, CH2 and CH3 regions)
an IgG light chain (consisting of VL and CL regions), and/or
a Fab and/or F(ab)₂.

As used herein, the term “derivative” shall refer to protein constructs being structurally different from, but still having some structural relationship to, the common antibody concept, e.g., scFv, Fab and/or F(ab)₂, as well as bi-, tri- or higher specific antibody constructs. All these items are explained below.

Methods for the production and/or selection of chimeric, humanised and/or human mAbs are known in the art. For example, U.S. Pat. No. 6,331,415 by Genentech describes the production of chimeric antibodies, while U.S. Pat. No. 6,548,640 by Medical Research Council describes CDR grafting techniques and U.S. Pat. No. 5,859,205 by Celltech describes the production of humanised antibodies. In vitro antibody libraries are, among others, disclosed in U.S. Pat. No. 6,300,064 by MorphoSys and U.S. Pat. No. 6,248,516 by MRC/Scripps/Stratagene. Phage Display techniques are for example disclosed in U.S. Pat. No. 5,223,409 by Dyax. Transgenic mammal platforms are for example described in US200302048621 by TaconicArtemis.

IgG, scFv, Fab and/or F(ab)₂ are antibody formats well known to the skilled person. Related enabling techniques are available from the respective textbooks.

As used herein, the term “Fab” relates to an IgG fragment comprising the antigen binding region, said fragment being composed of one constant and one variable domain from each heavy and light chain of the antibody

As used herein, the term “F(ab)₂” relates to an IgG fragment consisting of two Fab fragments connected to one another by disulfide bonds.

As used herein, the term “scFv” relates to a single-chain variable fragment being a fusion of the variable regions of the heavy and light chains of immunoglobulins, linked together with a short linker, usually serine (S) or glycine (G). This chimeric molecule retains the specificity of the original immunoglobulin, despite removal of the constant regions and the introduction of a linker peptide.

The term “new antibody formats” encompasses, for example bi- or trispecific antibody constructs, Diabodies, Camelid Antibodies, Domain Antibodies, bivalent homodimers with two chains consisting of scFvs, IgAs (two IgG structures joined by a J chain and a secretory component), shark antibodies, antibodies consisting of new world primate framework plus non-new world primate CDR, dimerised constructs comprising CH3+VL+VH, and antibody conjugates (e.g., antibody or fragments or derivatives linked to a toxin, a cytokine, a radioisotope or a label). This list is however not restrictive.

Further, the term also encompasses immunotoxins, i.e., heterodimeric molecules consisting of an antibody, or a fragment thereof, and a cytotoxic, radioactive or apoptotic factor. Such type of format has for example been developed by Philogen (e.g., anti-EDB human antibody L19, fused to human TNF), or Trastuzumab emtansine (T-DM1), which consists of trastuzumab linked to the cytotoxoic Mertansine (DM1).

As the inventors of the present invention have shown that L-Kynurenine, 3HAA, Cinnabarinic acid are overabundant in tumor tissue, targeting these metabolites with a specific immunotoxin represents a very promising therapeutic approach of site-directed tumor therapy.

The term “fusion peptide” or “fusion protein” proteins relates, for example, to proteins consisting of an immunoglobulin Fc portion plus a target binding moiety capable of binding an enzyme and/or a metabolite of the kynurenine pathway (so-called -cept molecules).

The term “antibody mimetic” relates to target binding proteins, which are not related to immunoglobulins. Many of the above mentioned techniques, like phage display, are applicable for these molecules as well. Such antibody mimetics are for example derived from Ankyrin Repeat Proteins, C-Type Lectins, A-domain proteins of Staphylococcus aureus, Transferrins, Lipocalins, Fibronectins, Kunitz domain protease inhibitors, Ubiquitin, Cysteine knots or knottins, thioredoxin A, and so forth, and are known to the skilled person in the art from the respective literature.

The term “aptamer”, as used herein, relates to nucleic Acid species, which are capable of binding to molecular targets such as small molecules, proteins, nucleic Acids, and even cells, tissues and organisms. Aptamers are useful in biotechnological and therapeutic applications as they offer molecular recognition properties that rival that of the commonly used biomolecule, antibodies. In addition to their discriminate recognition, aptamers offer advantages over antibodies or other target binders as they can be engineered completely in a test tube, are readily produced by chemical synthesis, possess desirable storage properties, and elicit little or no immunogenicity in therapeutic applications. Aptamers can for example be produced through repeated rounds of in vitro selection or equivalently, SELEX (systematic evolution of ligands by exponential enrichment) to bind

The term “small molecule antagonist”, as used herein, relates to a low molecular weight organic compound, which is by definition not a polymer. The term small molecule, especially within the field of pharmacology, is usually restricted to a molecule that also binds with high affinity to a biopolymer such as protein, nucleic Acid, or polysaccharide and in addition alters the activity or function of the biopolymer. The upper molecular weight limit for a small molecule is often set at 800 Daltons, which allows for the possibility to rapidly diffuse across cell membranes so that they can reach intracellular sites of action. In addition, this molecular weight cutoff is a necessary but insufficient condition for oral bioavailability. Small molecules acting as antagonists against a given target, e.g., an enzyme and/or a metabolite of the kynurenine pathway, can be found by high throughput screening of respective libraries comprising a large variety of different small molecular candidates.

In another preferred embodiment, said modulator and/or antagonist to an enzyme and/or a metabolite of the kynurenine pathway is conjugated to a given carrier.

Said conjugation to a given carrier may serve to increase the bioavailability, the efficiency and/or the serum half-life of the modulator and/or antagonist according to the invention.

One example for such conjugate is a PEGylated antibody fragment. PEGylation involves the modification of a protein, peptide, or non-peptide molecule by linking of one or more polyethylene glycol chains to it, and thus results in a prolonged serum half-life particularly of smaller protein drugs, like antibody fragments, as for example put into practice in the pegylated Fab fragment Certolizumab pegol.

A similar effect is obtained by N-glycosylation of respective domains in a given protein therapeutic (see above definition). Preferably, additional N-glycosylation sites are introduced into said protein therapeutic. This can be done by introducing, for example by site-directed mutagenesis, or by deliberate exchange of amino Acid residues, additional N-glycosylation motifs, i.e., tripeptide sequences Asn-X-Ser or Asn-X-Thr, where X can be any amino Acid (although Pro and Asp are rarely found). If for example the antibody, or fragment or derivative thereof, has, somewhere in its chain, the motif “Gly-X-Ser”, one could substitute “Gly” by “Asn”, on order to create an additional N-glycosylation site. It is of course necessary to make sure that the said substitution does not affect important properties of the protein, like target affinity, binding by Fc gamma receptors (FcyRs) or the like.

Increasing half-life can further be obtained by conjugating the said antagonist to an enzyme and/or a metabolite of the kynurenine pathway to a polypeptidic carrier such as Poly-L-Lysine or modified Poly-L-Lysine. This method encompasses the covalent binding of the antagonist to Poly-Lysine and/or modified Poly-L-Lysine. Modified Poly-L-Lysine can be obtained by adding at least one other moiety.

Conjugating the antagonist to Poly-Lysine can be performed by using crosslinkers that react with the free amine group of the Poly-L-Lysine and reactive functions on the antagonist. Grafting the antagonist on modified poly-l-lysine can be performed by crosslinking of the reactive groupement of antagonist to new reactive functions of the Pol-L-Lysine. Crosslinkers can be for example glutaraldehyde (NH₂ to NH₂), EDC (NH₂ to COOH), SMCC (NH₂ to SH). This conjugate would be able to limit liver and kidney filtration of the therapeutic protein as well as limiting its degradation by specific enzymes and therefore increasing its efficiency.

In another embodiment of the invention, a combination preparation comprising at least (i) the modulator and/or antagonist to an enzyme and/or a metabolite of the kynurenine pathway according to any of the aforementioned claims and (ii) at least one more active substance selected from the group consisting of
a antineoplastic agent
a targeted drug,
an endocrine drug,
a tumor vaccine,
immunotherapy, and/or
cellular therapy
   is provided.

As used herein, the term “antineoplastic agent” relates to a drug, or a combination of drugs, which have antineoplastic or anticancer effects. This applies, above all, to chemotherapeutic agents, which work by impairing mitosis, effectively targeting fast-dividing cells, or by causing cells to undergo apoptosis. The majority of chemotherapeutic drugs can be divided into alkylating agents, antimetabolites, anthracyclines, plant alkaloids, topoisomerase inhibitors, and other antitumour agents.

Targeted drugs are a type of medication that blocks the growth of cancer cells by interfering with specific targeted molecules needed for carcinogenesis and tumor growth, rather than by simply interfering with rapidly dividing cells (e.g. with traditional chemotherapy). The main categories of targeted therapy are small molecules and monoclonal antibodies.

Small molecules falling under this definition encompass, but are not limited, to Imatinib, Gefitinib, Erlotinib, Bortezomib, Bcl-2 inhibitors (e.g. Obatoclax, ABT-263, and Gossypol), PARP inhibitors (e.g. Iniparib, Olaparib), Janus kinase inhibitors, PI3K inhibitors, Apatinib, AN-152, Doxorubicin linked to [D-Lys(6)]-LHRH, Pegaptanib, Sunitinib, Sorafenib, Tivozanib and Pazopanib. Monoclonal antibodies falling under this definition encompass, but are not limited, to Rituximab, Trastuzumab, Cetuximab and Bevacizumab

Endocrine drugs, as used herein, are drugs that are antagonistic to hormones or hormone receptors and thus interfere with cancer types that require hormones to grow. One example for such Endocrine drug is Tamoxifen, which is an antagonist of the estrogen receptor in breast tissue.

The term “cellular therapy”, as used herein, shall relate to cell-based therapies such as adoptive transfer of modified, or unmodified, cytotoxic lymphocytes or dendritic cells.

The term“tumor vaccine”, as used herein, refers to vaccines that either a) prevent infections with cancer-causing viruses (mode of action is similar to other vaccines against viral infections), b) treat existing cancer (therapeutic cancer vaccines) or c) prevent the development of cancer, or ameliorate its effects (prophylactic cancer vaccines).

One approach to produce a tumor vaccine of type b) or c) (also called “immunotherapeutic” herein) is to isolate proteins from cancer cells and immunize cancer patients against those proteins, in the hope of stimulating an immune reaction that would kill the cancer cells. Another approach to therapeutic anti-cancer vaccination is to generate the immune response in situ in the patient. This enhances the anti-tumor immune response to tumor antigens released following lytic virus replication providing an in situ, patient specific anti-tumor vaccine as a result. Yet another approach is to immunize the patient with a compound that plays a physiological role in cancer genesis, so that the human body eliminates said compound. In such case, the compound is a self-antigen or self hapten, i.e., it does not provoke a strong immune response when administered to the patient. It has thus to be conjugated to a given carrier.

In another embodiment of the invention, the use of the modulator and/or antagonist to an enzyme and/or a metabolite of the kynurenine pathway according to any of the aforementioned claims for the treatment of a neoplastic disease is provided.

Preferably, said use is complemented, in a coordinated fashion, by the administration of at least one active substance selected from the group consisting of
a antineoplastic agent
a targeted drug,
an endocrine drug,
a tumor vaccine,
immunotherapy, and/or
cellular therapy

The term “complemented, in a coordinated fashion”, as used herein, shall refer to a coadministration, which is carried out under a given regimen. This includes synchronous administration of the different compounds as well as time-shifted administration of the different compounds (e.g., compound A is given once and compound B is given several times thereafter, or vice versa, or both compounds are given synchronously and one of the two is also given at later stages).

In another preferred embodiment, said use is complemented, in a coordinated fashion, by at least one other treatment selected from the group consisting of
radiotherapy
surgery
laser ablation

The term “complemented, in a coordinated fashion”, as used herein, has the same meaning as set forth above.

In another embodiment of the invention, the use of a modulator and/or antagonist to an enzyme and/or a metabolite of the kynurenine pathway according to any of the aforementioned claims in the diagnosis, prognosis, risk assessment and/or prediction of a neoplastic disease is provided.

The inventors have provided evidence that the presence of an enzyme and/or a metabolite of the kynurenine pathway in a given sample has a diagnostic, prognostic and/or predictive content with regard to neoplastic diseases. Such approach can be carried out with standard diagnostic methods, like Immunohistochemistry (IHC) or standard ELISA (Enzyme linked Immuno Assay) or advanced EIA (Enzyme Immuno Assay, a technology using a tracer compound) or other immunohistochemical or immunodiagnostical methods. Besides what is disclosed in this specification, the skilled person will find, in the suitable textbooks, a wealth of information on how to put this concept into practice.

Preferably, said modulator and/or antagonist to an enzyme and/or a metabolite of the kynurenine pathway is labeled.

Such label is, preferably, selected from the group consisting of a Radiolabel, Fluorescent label, a Luminescent label and/or an enzyme label. Said labeling can be direct, i.e., the antagonist itself is labeled. As an alternative, the labeling can be indirect (e.g., by means of a labeled secondary antibody which detected the antagonist antibody).

Furthermore, the use of an enzyme and/or a metabolite of the kynurenine pathway for the development of a modulator and/or an antagonist against said enzyme and/or metabolite is provided, said modulator and/or antagonist being useful as a therapeutic, and/or a diagnostic agent.

Generally, the development of an antagonist against a novel, well described moiety, be it a small molecule (like a metabolite) or a protein (like an enzyme), is within what the skilled person would consider as routine. The respective toolbox (conjugation to a carrier, immunization experiments, hybrodima technologies, affinity maturation, chimerization, humanization, display technologies, high throughput screening and the like) is readily available to the skilled person.

Preferably, said metabolite of the kynurenine pathway is 3HAA, L-Kynurenine, Quinolinic Acid and/or Cinnabarinic Acid. Also preferably, said enzyme of the kynurenine pathway is Kynureninase. More preferably, said modulator and/or antagonist is at least one modulator and/or antagonist according to the present invention.

It is further preferred that said development of a modulator and/or antagonist comprises at least one step of screening at least one library against the enzyme and/or a metabolite of the kynurenine pathway.

Such library can be an antibody library, e.g., as it is used for phage display or retrocyte display (see e.g. Hogenboom 2005). Such library can however also be a small molecule library, as e.g. described by Inglese et al (2007).

In another embodiment of the present invention a method of treatment of a neoplastic disease in a human or animal patient is provided, which method comprises the modulation of at least one parameter selected from the group consisting of the formation, concentration, availability and/or effect of 3HAA (3-Hydroxy Anthranilic Acid), L-Kynurenine, Quinolinic Acid and/or Cinnabarinic Acid, and/or Kynureninase.

Preferably, said method comprises the administration of a modulator and/or antagonist according to the present invention

In still another embodiment of the present invention a method of diagnosis, prognosis, risk assessment and/or prediction of a physiological and/or pathological condition, is provided, in which method the presence and/or concentration of an enzyme and/or a metabolite of the kynurenine pathway, either in free form and/or in form of the conjugated pool thereof, in a given sample is determined

As used herein, the term “conjugated pool” relates to enzymes and/or a metabolites which are conjugated to other entities, i.e., to ubiquitin, to enzymes, to receptors or the like. Thus, the the conjugated pool relates to any form of the target enzyme and/or metabolite which is not free. Preferably, this can mean that in the conjugated pool the target is immunogenic enough to be detected by an antibody. Preferably, the physiological and/or pathological condition is a neoplastic disease.

Preferably, in said method the presence and/or concentration of an enzyme and/or a metabolite of the kynurenine pathway is determined in a tissue sample and/or in a liquid sample.

Said tissue sample is for example a tissue slice, or a homogenized sample from a biopsy. Said liquid sample is for example a urine sample, saliva sample, blood serum sample, blood plasma sample, feces sample, sweat sample, swab sample, smear sample, a cell culture supernatant or the like.

Likewise, a method of research or screening is provided, in which method the presence and/or concentration of an enzyme and/or a metabolite of the kynurenine pathway, either in free form and/or in form of the conjugated pool thereof, in a given sample is determined

The term research relates to basic, fundamental or applied research. The term screening relates to methods, in which large numbers of samples are screened, either for research purposes or for diagnosis or epidemiology.

In another preferred embodiment of said method, the presence and/or concentration an enzyme and/or a metabolite of the kynurenine pathway, either in free form and/or in form of the conjugated pool thereof, in a tissue sample and/or in a liquid sample is determined by at least one method selected from the group of
Immunohistochemistry, ELISA, EIA and/or Immunofluorescence
in situ PCR (e.g., in tissue slices)
realTime PCR (also called rT PCR, or quantitative PCR) (e.g., in homogenized/liquid samples)
Gas Chromatography/Mass Spectroscopy (GC/MS)
High Performance Liquid Chromatography (HPLC)
Liquid Chromatography/Mass spectroscopy (LC/MS)

Immunohistochemistry, ELISA, EIA and Immunofluorescence can be carried out on liquid samples, smears, biopsies, sections of tissue blocks, tissue microarrays. The quantification of the detected analytes can be carried out by using, e.g., microscopy, laser scanning cytometry or flow cytometry Immunohistochemistry, ELISA, EIA and Immunofluorescence can be used to detect enzymes and metabolites of the kynurenine pathway.

Preferably, in such method the presence and/or concentration of said enzyme and/or metabolite of the kynurenine pathway in a tissue sample and/or in a liquid sample is determined by at using at least one modulator and/or antagonist according to the present invention according.

According to another preferred embodiment, of such method the enzyme and/or the metabolite of the kynurenine pathway is at least one selected from the group consisting of 3HAA, (3-Hydroxy Anthranilic Acid), L-Kynurenine, Quinolinic Acid, Cinnabarinic Acid, and/or Kynureninase.

Preferably, in such method a physiological and/or pathological condition is (i) diagnosed, (ii) prognosed, (iii) its risk is assessed, (iv) a prediction is made, wherein such condition is characterized by any form of activity of Indoleamine 2,3-dioxygenase 1 (IDO1), and/or Indoleamine 2,3 dioxygenase 2 (IDO2), and/or Tryptophan 2,3-dioxygenase (TDO2).

Alternatively, in such method the sample is characterized by any form of activity of Indoleamine 2,3-dioxygenase 1 (IDO1), and/or Indoleamine 2,3 dioxygenase 2 (IDO2), and/or Tryptophan 2,3-dioxygenase (TDO2).

As used herein, the term “characterized by any form of activity of IDO1, IDO2 and/or TDO2” relates to conditions in which the latter enzymes exert an activity which may lead to a pathological condition. This may be, for example, caused by overexpression, or by expression of a modified mutant, of IDO1, IDO2 and/or TDO2.

Besides what is disclosed in this specification, the skilled person will find, in the suitable textbooks, a wealth of information on how to put the concepts of molecular diagnosis (like for example in situ PCR or realTime PCR) or classical analytics (like for example GC/MS, HPLC, or LC/MS) into practice.

### EXPERIMENTS AND FIGURES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Antibodies Strategy

To limit the activity of exemplary metabolites of the kynurenine pathway, i.e., 3HAA, L-Kynurenine, Quinolinic Acid and/or Cinnabarinic Acid, we decided to use for the first time a specific antibody directed against these metabolites. The use of antibodies is a growing strategy in therapeutic field with several applications. It is important to point that all antibodies used in clinic are directed against peptides or proteins.

### 1. Synthesis of Specific Antibodies Directed Against Selected Metabolites of the Kynurenine Pathway.

The following section describes, in contrast thereto, the production of an antibody against a non-protein target, i.e., the small molecule 3HAA. The inventors have carried out such experiments also to create antibodies against other metabolites of the Kynurenine pathway, in particular against L-Kynurenine, Quinolinic Acid and/or Cinnabarinic Acid, but to avoid repetitions only the production of anti 3HAA antibodies is described in the following.

The skilled person will be able to transfer this teaching to other metabolites of the Kynurenine pathway, in particular against L-Kynurenine, Quinolinic Acid and/or Cinnabarinic Acid.

In an attempt to make antibodies against a small molecule target one first needs to prepare the immunogen by crosslinking the small molecule to an immunogenic carrier. The different possibilities to couple a small molecule (also called hapten) or a self antigen to a carrier in order to make it immunogenic are discussed below.

In the present experiment, 3HAA was conjugated to bovine serum albumine (BSA) by means of a carbodiimide crosslinker, which reacts with the carboxylic function of 3HAA and free amine functions of the BSA, to form a stable amide bond. The “Imject BSA and EDC Conjugation Kit” provided by Thermo Scientific was used for this purpose.

### 1.1 Production of Monoclonal Antibodies

Monoclonal antibodies were generated following the established method of Köhler and Milstein (Köhler & Milstein 1975). Briefly, lymphocytes were isolated from mice immunized three times with the 3HAA-BSA conjugates. The lymphocytes were then fused with murine myeloma cells (SP2O-Ag) with polyethyleneglycol (PEG 1500) to obtain hybridoma cells. The selection of hybridomas cells was realized by enzyme-linked immunosorbent assay (ELISA).

Clone 5B2-G2 was selected as the most promising clone. Monoclonal antibodies to 3HAA produced by Clone 5B2-G2 had an affinity of 10⁻¹⁰ M (calculated based on the conjugates amount used to make the competition assay in ELISA, wherein the amount of conjugates is related to amount of BSA). No cross reactions with other metabolites from the kynurenines pathway could be found.

### 1.2 Deposit of Clone 1B10 Hybridoma Cells Producing the Selected Anti 3HAA Antibody

The Clone 1B10 hybridoma cells have been deposited on May 31, 2012 at the “Collection Nationale de Culture de Microorganismes” (CNCM) at Pasteur Institute (25 rue du Docteur Roux F-75724 PARIS Cedex 15). The deposit has been made by means of 12 cryotubes containing more than 1*10⁶ units as specified. The deposit number is CNCM 1-4637.

### 1.3. Other Clones

Other hybridoma cell clones developed in the context of the present invention produce monoclonal antibodies against 3HAA, Kynurenine, Cinnabarinic Acid and Quinolinic Acid. These clones are deposited in the laboratory of the inventors. 10 clones have been isolated which produce monoclonal antibodies against 3HAA, 3 clones have been isolated which produce monoclonal antibodies against Kynurenine, 3 clones have been isolated which produce monoclonal antibodies against Cinnabarinic Acid, and 5 clones have been isolated which produce monoclonal antibodies against Quinolinic Acid

| Target | Clone Names |
|---|---|
| 3HAA | 6A10-B9, 5G-D11, 6A1-F2, 6F6-A2, 4A5-H9, |
| | 1A10-D11, 6C4-H9, 5B2-G2, 1B10, 2A12 |
| Kynurenine | 5C1-G5, 3D4-F2, 2E6-F2 |
| Cinnabarinic Acid | 6D3-A7, 7C7-A2, 5C5-E10 |
| Quinolinic Acid | 1A6-F6, 4E11-G3, 3C10-E5, 1H1-E3, 3B2-C7 |

### 2. ELISA Competition Assay

To characterize the monoclonal antibodies, their affinities were evaluated in an ELISA competition assay. Briefly, maxisorp 96 well-plates (Nunc) were coated with the respective conjugated antigen (e.g., 3HAA-BSA for an antibody against the same conjugate) in carbonate buffer (pH=9.6) overnight at 4° C. After saturation with PBS-Tween 0.01%+BSA 0.25% (ELISA Buffer) for 1 h at 37° C., plates were washed three times with PBS-T (PBS+Tween 0.01%) and antibodies were added. Antibodies were previously incubated with increasing concentration of the respective conjugate or its competitor in ELISA buffer for 1 h30 at 37° C. Three washes in PBS-T were performed and anti mouse IgG (Abliance) were added for 1 hour at 37° C. The solution was then removed, plates washed three times and revelation acetate buffer plus tetratmethylbenzidine allowed antibodies detection. Optical density (OD) was evaluated at 450 nm. Results were represented by the ratio B/B0 where B0 is the OD obtained with the antibodies alone (without any antigen) and B the OD obtained for a specific antigen at a specific concentration. More the antibodies is affine, less conjugate will be necessary to decrease the OD value. For a specific antibody, the ratio B/B0 should not change when the antibodies is incubated with a competitor conjugate.

### 3. Enzyme Immunoassay (EIA)

### 3.1. Detection of the Conjugated Pool of Kynurenine by EIA

EIA is different from the classic ELISA since a tracer is used. By tracer we mean the Kynurenine conjugated to Horseradish peroxidase (HRP). Briefly, maxisorp 96 well-plates (Nunc) were coated with anti mouse IgG (Abliance) in carbonate buffer (pH=9.6) overnight at 4° C. After saturation with PBS-Tween 0.01%+BSA 1% (EIA Buffer) for 1 h at 37° C., plates were washed three times with PBS-T and antibodies for 1 h30 at 37° C. 3D4-F2 antibodies were previously incubated at 0.01 mg/ml with increasing concentration of the respective conjugate in EIA buffer for 1 hour at 37° C. Then, a tracer was added to the solution at a final concentration of 1 μg/ml. Four washes in PBS-T were performed and revelation acetate buffer plus tetratmethylbenzidine allowed antibodies detection. Optical density (OD) was evaluated at 450 nm. Results were represented by the ratio B/B0, where B0 is the OD obtained with 3D4-F2 and the Kynurenine-HRP alone (without the conjugate) and B the OD obtained at a specific concentration. The affinity was evaluated at 10⁻⁹M.

### 3.2. Detection of the Free Pool of Kynurenine

To allow the detection of the free pool of a small and non-immunogenic molecule a derivatization step is necessary to make the molecule reacting with amine containing molecules (eg proteins). To mimic a biological fluid (eg sera), we prepared a solution containing 70 mg/ml of protein. Solutions containing different amount of L-Kynurenine were prepared in this buffer. Then, the derivatization process was performed using carbodiimide (EDC) plus N-hydroxysuccinimide (NHS) in MES buffer for 1 hour at 37° C. The reaction was stopped by addition of glycine and this final solution was incubated with the 3D4-F2 antibodies at 0.01 mg/ml for 1 h at 37° C. The HRP-Kynurenine was then added at 1 μg/ml and the antibodies solution containing the derivatization product plus the tracer was incubated for 1 h30 on the plate. Four washes in PBS-T were performed and revelation acetate buffer plus tetramethylbenzidine allowed antibodies detection. Optical density (OD) was evaluated at 450 nm. Results were represented by the ratio B/B0 where B0 is the OD obtained with the 3D4-F2 and the Kynurenine-HRP alone (without derivatization product) and B the OD obtained at a specific concentration. The affinity for the free L-Kynurenine was estimated at 5*10⁻⁶M.

### 3.3. Detection of the Conjugated Pool of 3HAA by EIA

Here, by tracer we mean the 3HAA conjugated to Horseradish peroxidase (HRP). Briefly, maxisorp 96 well-plates (Nunc) were coated with anti mouse IgG (Abliance) in carbonate buffer (pH=9.6) overnight at 4° C. After saturation with PBS-Tween 0.01%+BSA 1% (EIA Buffer) for 1 h at 37° C., plates were washed three times with PBS-T and antibodies for 1 h30 at 37° C. 5B2-G2 antibodies were previously incubated at 0.01 mg/ml with increasing concentration of the respective conjugate in EIA buffer for 1 hour at 37° C. Then, a tracer was added to the solution at a final concentration of 1 μg/ml. Four washes in PBS-T were performed and revelation acetate buffer plus tetramethylbenzidine allowed antibodies detection. Optical density (OD) was evaluated at 450 nm. Results were represented by the ratio B/B0, where B0 is the OD obtained with 5B2-G2 and the 3HAA-HRP alone (without the conjugate) and B the OD obtained at a specific concentration. The affinity was evaluated at 5*10⁻¹° M.

### 3.4. Detection of the Free Pool of 3HAA

Solutions containing different amount of 3HAA were prepared in Dimethylsulfoxide supplemented with triethylamine (TEA) to increase the pH. Then, the derivatization process was performed using ethylchloroformate (ECF) diluted in Dimethylformamide (DMF) for 10 minutes at room temperature. Then, the reaction product was added drops by drops to 2 mg of BSA diluted in alkaline water (using TEA). This final solution was incubated with the 5B2-G2 antibodies at 0.01 mg/ml for 1 h at 37° C. The HRP-3HAA was then added at 1 μg/ml and the antibodies solution containing the derivatization product plus the tracer was incubated for 1 h30 on the plate. Four washes in PBS-T were performed and revelation acetate buffer plus tetratmethylbenzidine allowed antibodies detection. Optical density (OD) was evaluated at 450 nm. Results were represented by the ratio B/B0 where B0 is the OD obtained with the 5B2-G2 and the 3HAA-HRP alone (without derivatization product) and B the OD obtained at a specific concentration. The affinity for the free 3HAA was estimated at 5*10⁻⁴M.

### 3.5. Detection of the Conjugated Pool of Cinnabarinic Acid by EIA

Here, by tracer we mean the Cinnabarinic Acid conjugated to Horseradish peroxidase (HRP). Briefly, maxisorp 96 well-plates (Nunc) were coated with anti mouse IgG (Abliance) in carbonate buffer (pH=9.6) overnight at 4° C. After saturation with PBS-Tween 0.01%+BSA 1% (EIA Buffer) for 1 h at 37° C., plates were washed three times with PBS-T and antibodies for 1 h30 at 37° C. 7C7-A2 antibodies were previously incubated (after 10 times dilution of cell culture supernatant) with increasing concentration of the respective conjugate in EIA buffer for 1 hour at 37° C. Then, a tracer was added to the solution at a final concentration of 0.03 μg/ml. Four washes in PBS-T were performed and revelation acetate buffer plus tetratmethylbenzidine allowed antibodies detection. Optical density (OD) was evaluated at 450 nm. Results were represented by the ratio B/B0, where B0 is the OD obtained with 7C7-A2 and the CA-HRP alone (without the conjugate) and B the OD obtained at a specific concentration. The affinity was <5*10⁻¹¹M.

### 3.6. Detection of the Free Pool of Cinnabarinic Acid (CA)

Solutions containing different amount of CA were prepared in PBS+BSA 70 g/l. Then, the derivatization process was performed using carbodiimide (EDC) plus N-hydroxysuccinimide (NHS) in MES buffer for 1 hour at 37° C. The reaction was stopped by addition of glycine and this final solution was incubated with the 7C7-A2 (after 10 times dilution of the cell culture supernatant) for 1 h at 37° C. The HRP-CA was then added at 0.03 μg/ml and the antibodies solution containing the derivatization product plus the tracer was incubated for 1 h30 on the plate. Four washes in PBS-T were performed and revelation acetate buffer plus tetratmethylbenzidine allowed antibodies detection. Optical density (OD) was evaluated at 450 nm. Results were represented by the ratio B/B0 where B0 is the OD obtained with the 7C7-A2 and the CA-HRP alone (without derivatization product) and B the OD obtained at a specific concentration. The affinity for the free CA was <10⁻⁷M.

### 4. Immunohistochemistry

### 4.1. Detection of L-Kynurenine in Human Colon Tumours

Human colon tumours were purchased from USBiomax (T054a) as Tissue Micro Array with 10 cases harboring colon carcinomas and 2 healthy subjects. Experimentally, sections were deparafinized using successive bath of Xylene and Ethanol. Sections were then subjected to antigen retrieval with citrate buffer pH=6 (Dako) for 20 minutes at 95° C. Sections were washed in TBS before incubation with methanol containing 0.03% of hydrogen peroxide to block endogenous peroxydase. After two washes, sections were saturated in antibody diluent (Dako) plus 5% of BSA (Sigma-Aldrich) for 30 minutes at room temperature.

Anti L-Kynurenine mAb (3D4-F2) was then added at 0.01 mg/ml, in the presence of 2% of normal goat serum, and incubated overnight at 4° C. Sections were washed three times in TBS, and incubated for 30 minutes with envision system (dextran polymer grafted with anti mouse IgG conjugated with HRP, Dako) at room temperature. Sections were washed three times before revelation with DAB (Dako) for 10 minutes at room temperature.

Sections were rinsed, subjected to hematoxylin, dehydrated and mounted in DPX mountant media (Sigma-Aldrich). Pictures were obtained after a systematic scan of all cores (TissueGnostics).

Quantification was performed according to the following grades:
0: No staining
1: Weak staining
2: Intermediate staining
3: Strong staining

In these samples, 40% of patients show a strong staining, 10% of patients an intermediate staining, 20% of patients a weak staining and 30% of patients an absence of staining. These data reveal that L-Kynurenine production pattern, revealed by IHC, can discriminate patients with colon tumours.

### 4.2. Detection of L-Kynurenine in Human Breast Tumours

The same experimental setting was used to evaluate the production of L-Kynurenine in human breast tumours. Samples were obtained from USBiomax (T086c) as Tissue Micro Array with 10 cases of breast carcinomas and 2 healthy subjects.

In these samples, 10% of patients show a strong staining, 10% of patients an intermediate staining, 40% of patients a weak staining and 40% of patients an absence of staining. These data reveal that L-Kynurenine production pattern, revealed by IHC, can discriminate patients with breast tumours.

### 4.3. Detection of 3HAA in Human Colon Tumours

The same human colon tumours Tissue Micro Array (T054a) was used to evaluate the 3HAA production. Experimentally, sections were deparafinized using successive bath of Xylene and Ethanol. Sections were then subjected to antigen retrieval with citrate buffer pH=6 (Dako) for 20 minutes at 95° C. Sections were washed in TBS before incubation with methanol containing 0.03% of hydrogen peroxide to block endogenous peroxydase. After two washes, sections were saturated in antibody diluent (Dako) plus 5% of BSA (Sigma-Aldrich) for 30 minutes at room temperature. Anti 3HAA mAb (5B2-G2) was then added at 0.001 mg/ml, in the presence of 2% of normal goat serum, and incubated overnight at 4° C. Sections were washed three times in TBS, and incubated for 30 minutes with envision system (dextran polymer grafted with anti mouse IgG conjugated with HRP, Dako) at room temperature. Sections were washed three times before revelation with DAB (Dako) for 10 minutes at room temperature. Sections were rinsed, subjected to hematoxylin, dehydrated and mounted in DPX mountant media (Sigma-Aldrich). Pictures were obtained after a systematic scan of all cores (TissueGnostics).

Quantification was performed according to the following grades:
0: No staining
1: Weak staining
2: Intermediate staining
3: Strong staining

In these samples, 60% of patients show an intermediate staining, 20% of patients low staining and 20% of patients an absence of staining. These data reveal that 3HAA production pattern, revealed by IHC, can discriminate patients with colon tumours

### 4.4. Detection of 3HAA in Human Breast Tumours

The same experimental setting was used to evaluate the production of 3HAA in human breast tumours. Samples were the same as for the L-Kynurenine (USBiomax, T086c).

In these samples, 20% of patients shows a strong staining, 50% of patients an intermediate staining, 30% of patients a weak staining These data reveal that 3HAA production pattern, revealed by IHC, can discriminate patients with breast tumours.

### 4.5. Detection of 3HAA in a Mouse Model of Glioblastoma

The same experimental setting was used to evaluate the production of 3HAA a mouse model of intracerebral glioblastoma. This model was obtained by intracerebral injection of GL261 to immunocompetent mice. After 29 days, brains were taken, and prepared for immunohistochemistry.

In these samples, 3HAA was detectable in tumour cells but also in reactive surrounding astrocytes (see FIG. 15)

### 4.6. Detection of Cinnabarinic Acid in Human Colon Tumours

The same human colon tumours Tissue Micro Array (T054a) was used to evaluate the CA production. Experimentally, sections were deparafinized using successive bath of Xylene and Ethanol. Sections were then subjected to antigen retrieval with citrate buffer pH=6 (Dako) for 20 minutes at 95° C. Sections were washed in TBS before incubation with methanol containing 0.03% of hydrogen peroxide to block endogenous peroxydase. After two washes, sections were saturated in antibody diluent (Dako) plus 5% of BSA (Sigma-Aldrich) for 30 minutes at room temperature. Anti Cinnabarinic Acid mAb (5C5-E10) was then added at 0.05 mg/ml, in the presence of 2% of normal goat serum, and incubated overnight at 4° C. Sections were washed three times in TBS, and incubated for 30 minutes with envision system (dextran polymer grafted with anti mouse IgG conjugated with HRP, Dako) at room temperature. Sections were washed three times before revelation with DAB (Dako) for 10 minutes at room temperature. Sections were rinsed, subjected to hematoxylin, dehydrated and mounted in DPX mountant media (Sigma-Aldrich). Pictures were obtained after a systematic scan of all cores (TissueGnostics).

Quantification was performed according to the following grades:
0: No staining
1: Weak staining
2: Intermediate staining
3: Strong staining

In these samples, 40% of patients show a strong staining, 10% of patients an intermediate staining, 20% of patients a weak staining and 30% of patients an absence of staining. These data reveal that CA production pattern, revealed by IHC, can discriminate patients with colon tumours.

### 4.7. Detection of Cinnabarinic Acid in Human Breast Tumours

The same experimental setting was used to evaluate the production of Cinnabarinic in human breast tumours. Samples were the same as for the L-Kynurenine and 3HAA (USBiomax, T086c).

In these samples, 20% of patients show a strong staining, 10% of patients an intermediate staining, 50% of patients a weak staining and 20% of patients an absence of staining. These data reveal that CA production pattern, revealed by IHC, can discriminate patients with breast tumours.

### 5. In-Vitro Effects

### 5.1 In Vitro Activity of the Selected Anti 3HAA Antibody

We evaluated the ability of the selected anti 3HAA antibody to block the suppressive effect of 3HAA on helper T cells activated with CD3/CD28 cocktail antibodies. Helper T cells were incubated with or without 3HAA and stained with CFSE (carboxyfluorescein succinimidyl ester). As expected, we observed that 3HAA blocked cell proliferation of T cells. This effect was largely suppressed when the 3HAA antibody (1B10) was added to culture medium (FIG. 21). The selected anti 3HAA antibody is therefore able to inhibit, in vitro, the suppressive effect of 3HAA, e.g. on helper T cells.

### 5.2 In Vitro Effect of the Selected Anti Cinnabarinic Acid Antibody

We evaluated the ability of the selected antibody to modulate the cell proliferation rate of 2 human colorectal cancer cell lines, HT29 (ATCC-HTB38) and HCT116 (ATCC-CCL247). For that purpose, cells were plated on a 24 well/plate at 5*104 cells/wells for 24 hours. Cells were then incubated with a culture supernatant of our 7C7-A2 hybridoma for 48 hours. A blind cell-count was then performed using a Malassez cell. These data show a substantial decrease in HT29 proliferation while only a slight decrease was observes in HCT116 cells (FIG. 22). These data provide for the first time that targeting one metabolite of the Kynurenine pathway, could affect the proliferation rate of a human cancer cell lines and particularly a colorectal cancer cell line.

### 6. In Vivo Properties of the Selected Anti 3HAA Antibody

### 6.1. Melanoma Model

Melanoma is well known to involve immune escape. We thus evaluated the benefits of IgG anti 3HAA in an experimental model of melanoma. This model was obtained by subcutaneous implantation of 5*10⁴ B16-F10 cells (ATCC CRL-6475) in immunocompetent C57BL/6 mice. Tumour size was followed for 22 days after treatment with either vehicle, IgG anti 3HAA (1B10) or Dacarbazine. 100 μg of IgG anti 3HAA were administered subcutaneously at day 6 (at a time when tumours were detectable), 13 and 20. Dacarbazine was administered intra-peritonealy at 80 mg/kg. The treatment started at day 6 and was repeated once every 2 weeks for 4 consecutive times; chemotherapy cycles were repeated every 4 days. These results show a substantial benefit of the IgG anti 3HAA with higher efficacy when compared to dacarbazine.

### 6.2. Glioblastoma Model (Subcutaneous)

We then evaluated benefits of the antibody in an experimental model of ectopic glioblastoma. Glioblastoma cells were implanted subcutaneously into C57BL/6J mice to follow the tumor growth. The selected anti 3HAA antibody was injected following two protocols: preventive and curative.

For the preventive treatment, 50 μg (low amount) of antibody was injected subcutaneously one day before cell injection. This treatment was repeated once weekly. Antibody administration resulted in a significant decrease of tumor growth (FIG. 15)

For the curative treatment, the 3HAA antibody was administered six days after cell injection (50 μg/mice) and was repeated once weekly with the same amount. A significant decrease of tumor growth was observed (FIG. 16).

### 6.3. Glioblastoma Model (Intracerebral)

Since glioblastoma is an aggressive tumor without an adequate therapeutic solution, we decided to evaluate the benefits of our antibody in an intracerebral (orthotopic) model of glioblastoma. We started the injection of the antibody (100 μg/mice) subcutaneously six days after cell implantation (line GL261 from ATCC) into striatum (0.1 mm posterior to the bregma and 2.3 mm lateral to the midline) of C57BL/6J mice. The administration was repeated once weekly. Mice were sacrificed 30 days after cell implantation, and brains were cutted (20 μm cryostat section) for tumor volume estimation.

All tumor sections were mounted on glass slides and pictures were taken to evaluate by software analysis (Metamorph) tumor surface on each slides. The total tumor volume was calculated by addition of all tumor surfaces. This analysis revealed a significant benefit of the administration of our antibody in an aggressive model of brain tumor (FIG. 17).

The 3HAA antibody possesses therefore significant anti-tumor properties when administered subcutaneously at low amounts.

### 7. Benefits of IgG Anti 3HAA/Kynurenine/Cinnabarinic Acid

### 7.1. Glioblastoma Model (Intracerebral)

Benefits of monoclonal antibodies directed against either L-Kynurenine (3D4-F2), 3HAA (1B10 and 5B2-G2) and Cinnabarinic Acid (5C5-E10) were evaluated in an intracerebral model of Glioblastoma obtained by GL261 tumour cells implantation into C57BL/6 mice. Briefly, mice were anesthetized under isoflurane gas and a burr hole was made in the skull at the bregma and 2.3 mm lateral to the midline. GL261 (5*10⁷/mL) were inoculated in 2 μL of saline supplemented with 4% FCS stereotactically (David Kopf Instruments) using a 10 μL Hamilton syringe. The needle was advanced to a depth of 2 3 mm from the brain surface and the cell suspension was delivered slowly over the course of 3 to 4 minutes. Following injection, the needle was left in place for 0.5 minutes. Animals were randomly assigned into control and treatment groups (n=8/group). Treatment started 6 days after cells implantation by subcutaneous injection of 100 μg of a specific IgG; the treatment was repeated once a week in the same condition.

30 Days after tumour cells implantation, 0% of mice treated with the vehicle survived while the % of survival protection was 12.5% for IgG anti 3HAA (1B10), 25% for IgG anti 3HAA (5B2-G2), 12.5% for IgG anti L-Kynurenine (3D4-F2) and 12.5% for IgG anti Cinnabarinic Acid (5C5-E10). These results show a benefit of targeting kynurenines pathway metabolites and particularly L-Kynurenine, 3HAA and Cinnabarinic Acid.

### 8. Production of Humanized Antibodies

The use of murine monoclonal antibodies in clinical settings is limited by the human anti-murine antibodies (HAMA) response against both variable and constant regions of the murine Abs (Reynolds et al. 1989). To circumvent this impediment, murine antibodies can be humanized. The first technology to address this need was the grafting of the complementary determining residues (CDRs) onto the variable light (VL) and variable heavy (VH) frameworks of human immunoglobulin molecules (Winter et al 1993). However, the remaining mice CDRs of the humanized antibodies can still generate an anti-idiotypic (anti-Id) response in patients. To limit the anti-Id response, only the specificity determining residues (SDRs), the most important CDR residues in the antibody-ligand binding are thus grafted.

The last method is used to generate a humanized anti 3HAA antibody. After some modifications in the sequence of the humanized antibody by affinity maturation (method according to Wu 2002) the humanized anti 3HAA antibody is able to recognize the conjugated 3HAA with the same affinity as the murine anti 3HAA antibody, and is still specific enough when tested against other metabolites of the kynurenine pathway.

The skilled person will understand that the above process can easily be modified in order to produce humanized antibodies against other metabolites of the Kynurenine pathway, like L-Kynurenine, Quinolinic Acid and/or Cinnabarinic Acid.

### 9. Production of a Fully Human Antibody in Transgenic Mice

Because a humanized antibody still possesses mice residues, new approaches have been developed to generate fully human antibodies by immunization of a transgenic mammal having the human immunoglobulin gene repertoire (Lonberg 2005). One example for such antibody is Panitumumab used in clinic.

For this purpose a transgenic mouse (HuMab™ mouse developed by medarex) is used. In this mouse, the endologoues immunoglobulin gene repertoire has been replaced by its human counterpart, so that, after immunization, said mouse produces fully-human antibodies.

In another approach, irradiated BALB/c Rag2−/−IL-2Rγc−/−mice reconstituted with human hematopoietic progenitor cells (hHPC) (Shultz et al 2007) are used.

In either case, the mice are immunized with 50 μg of 3HAA/BSA conjugates solubilised in 100 μl in NaCl 9 g/l and emulsified with 100 μl of Freund complete adjuvant (1st immunization) and in Freund incomplete adjuvant, for the 2nd and 3rd immunizations. Immunizations are repeated every 2 weeks. The mice are sacrificed 2 weeks after the 3^{rd} immunization and the spleens recovered and splenocytes are isolated. The latter are then fused with a human myeloma cell (Karpas 707, see Karpas et al. 2001) line to generate hybridoma cell lines. The selection of suitable hybridoma cell lines is performed by means of ELISA assay and the same procedure as used to generate the murine monoclonal antibody is followed. By this process, a clone can be obtained which produces a fully human IgG antibody with the same immunochemical properties as the murine anti 3HAA antibody discussed above

The skilled person will understand that the above process can easily be modified in order to produce fully human antibodies against other metabolites of the Kynurenine pathway, like L-Kynurenine, Quinolinic Acid and/or Cinnabarinic Acid.

### 10. Production of a Fully Human Antibody by Means of Phage Display

Methods to produce a human antibody against a hapten or self antigen by means of phage display are, e.g., discussed in Brichta et al. (2005), Kerrm et al. (2003), Keith et al. (2001) or Sheedy et al. (2007).

### 10.1. Preparation of Conjugates

3HAA is conjugated to both keyhole limpet haemocyanin (KLH) and bovine serum albumin (BSA) via linkage to 2-mercaptoethylamine, and hapten load/carrier protein is determined to be between eight and 10 haptens per BSA molecule using matrix assisted laser desorption spectrometry. The resulting conjugates are assayed for protein according to standard protocols.

### 10.2. Plasmids and Strains

The Griffin library (MRC Laboratories, Cambridge, UK) consists of the majority of human VH and VL chain gene segments used in vivo, with CDR3 diversity generated using synthetic oligonucleotides (semi-synthetic). The Tomlinson library (MRC Laboratories, Cambridge, UK) is based on a single human framework with side chain diversity (DVT encoded) incorporated at 18 amino Acid positions in the antigen binding site (synthetic). In both libraries, the antibodies are displayed as scFv fragments on the coat protein of filamentous bacteria in the phagemid vector pHEN II. The phagemid clones are maintained and propagated in T-phage resistant E. coli TG1Tr (Stratagene).

Antibody fragments are expressed using the dicistronic, expression vector pIMS147. The vector is inducible with isopropyl L-D-thiogalactosidase (IPTG) and downstream from the scFv genes contains a human CU domain (forming a single chain antibody or scAb) for immunodetection and a hexahistidine tail for purification by nickel chelate affinity chromatography. The antibody expression vector pIMS147 is maintained in E. coli strain XL-1 Blue (Stratagene).

### 10.3. Affinity Selection of Antibodies

One hundred microlitres of either Tomlinson or Griffin glycerol stock are inoculated into 100 ml 2×TY broth containing 1% glucose and 100 μg ml⁻¹ ampicillin (2×TY-glu-amp), and incubated with shaking at 37° C. to an OD 600 of 0.4. KM13 helper phage (2×10¹¹ pfu) are added to 50 ml of each library culture and the mixture incubated at 37° C. without shaking for 30 min.

Infected cells are pelleted, resuspended in 100 ml 2×TY broth-0.1% glu-amp-50 μg ml⁻¹ kanamycin, and incubated overnight with shaking at 30° C. Phage particles are concentrated from each culture supernatant by precipitation with 20 ml polyethylene glycol in 2.5 M NaCl (20% w/v) as described previously.

Two immunotubes are coated overnight with 100 μg ml⁻¹ 3 HAA-BSA in phosphate buffered saline (PBS), washed with PBS and blocked with 2% skimmed milk-PBS at room temperature for 2 h. The concentrated phage particles (approximately 1×10¹³) from each library (Griffin or Tomlinson) are added to the immunotubes. Specific scFv phage bound to the antigen, and the unbound phages are removed by washing. The bound scFv phage are eluted from the immunotube with triethylamine (TEA) and infected into exponential phase TG1 cell culture suspension in 2×TY broth for 30 min before being pelleted and plated onto agar plates of TYE-glu-amp and incubated at 30° C. overnight. The colonies are scraped into 5 ml of 2×TY-glu-amp-15% glycerol and stored at −80° C. Fifty microlitres of this stock are used to inoculate 50 ml fresh 2×TY-glu-amp and phage grown, infected and rescued as described above. Selection is repeated a further two times with the following modifications:

pan 2, 3HAA-KLH (100 μg ml⁻¹); pan 3, 3HAA-BSA (1 μg ml⁻¹).

### 10.4. Screening and Selection of Phage Antibodies

Phage antibody clones (phAbs) that only recognise 3HAA conjugates and not BSA or KLH alone are further characterised using a monoclonal binding ELISA where the phage antibodies are added to the plate in the presence or absence of free 3HAA.

Those phAbs showing reduction of binding compared with phAbs added to the plate alone are sequenced in both directions on an ABI377 automated DNA sequencer (P.E. Applied Biosystems, Foster City, Calif., USA). DNA from clones found to have different H or L chain sequences are digested with NcoI and NotI, and the scFv genes cloned into the similarly digested soluble expression vector pIMS147 before transformation into electrocompetent E. coli XL-1 Blue.

Antibodies are then identified which showed binding to 3HAA.

### 10.5. Large Scale Expression and Purification of Anti-Hapten Antibodies

Single E. coli XL-1 Blue colonies containing antibodies specific for 3HAA are grown overnight in 5 ml LB containing 1% (w/v) glucose, 50 μg ml⁻¹ amp and 12.5 μg ml⁻¹ tetracycline at 37° C. using published methods. Each culture is used to inoculate 50 ml Terrific broth (TB)-glu-amp-tet in 250-ml baffled flasks, and the culture is grown to an OD of 15. The cells are pelleted and resuspended in 50 ml fresh TB-amp before induction of antibody expression with IPTG (1 mM final concentration) for 4 h. The cells are pelleted, osmotically shocked and the supernatant containing the periplasmic fraction harvested, ready for purification.

The skilled person will understand that the processes discuss under items 10.1-10.5 can easily be modified in order to produce fully human antibodies against other metabolites of the Kynurenine pathway, like L-Kynurenine, Quinolinic Acid and/or Cinnabarinic Acid.

### 10.6. Synthesis of Specific Antibodies Directed Against Kynureninase

Kynureninase, or L-Kynurenine hydrolase, is a pyridoxal phosphate dependent enzyme that catalyses the formation of (i) Kynurenine into Anthranilic acid, and (ii) 3HAA from 3-Hydroxy Kynurenine. Humans express one kynureninase enzyme that is encoded by the KYNU gene located on chromosome 2. Kynureninase belongs to the class V group of aspartate aminotransferase superfamily of structurally homologous pyridoxal 5′-phosphate (PLP) dependent enzymes. To date, two structures of human kynureninase have determined by X-ray diffraction with resolutions of Forty percent of the amino acids are arranged in an alpha helical and twelve percent are arranged in beta sheets. Docking of the kynurenine substrate into the active site suggests that Asn-333 and His-102 are involved in substrate binding. Kynureninase has the ENZYME entry No EC 3.7.1.3. It has 465 AA residues and a molecular weight of about 52.4 kDa.

Antibodies against kynureninase can be produced with the established method portfolio known to the skilled person, e.g., by mere immunization of a mammal (polyclonal antibodies), the Kohler Milstein technique (Mouse monoclonal antibodies), Chimerization (Chimeric recombinant antibodies), CDR grafting (humanized antibodies), Affinity maturation and DNA shuffling, Phage display from a human library (human antibodies), Transgenic mammal techniques (human antibodies). See Lonberg 2005 for further reference.

### 11. Detection of Kynureninase in a Sample by Molecular Techniques

As set forth above, the presence of an enzyme and/or a metabolite of the kynurenine pathway may be useful in the diagnosis, prognosis, risk assessment and/or prediction of a neoplastic disease. This approach has been demonstrated by immunohistochemical detection of 3HAA in tumor tissue.

While 3HAA is a metabolite of the kynurenine pathway, enzymes of the kynurenine pathway can of course likewise be detected by means of immunohistochemistry. However, proteins, and particularly enzymes, can also be detected with modern molecular techniques, e.g., by detecting their mRNA in situ, or in a liquid sample. In the following, both approaches will be described for a selected enzyme of the kynurenine pathway, i.e., kynureninase. It is important to understand that this approach will also be applicable to other enzymes of the kynurenine pathway.

### 11.1. Detection of Kynureninase in a Sample by Real Time PCR

Primary human PBMC (peripheral blood mononuclear cells) are obtained from blood by Ficoll separation. Once separated, cells are subjected to lysis buffer (e.g., Trizol) and RNA is then extracted (follow classic protocol). After reverse transcription of the complete mRNA with a reverse transcriptase (Qiagen), the obtained cDNA are subjected to real time PCR (also termed quantitative PCR) with specific primers to human kynureninase (EC=3.7.1.3). The encoding sequence thereof can for example be found in the Uniprot database under entry No Q16719.

The design of specific primers for real time PCR is in the routine of the skilled person (see e.g., Pattyn et al. 2003) in case the nucleic acid sequence of the protein to be discovered is known. Major service providers have even online tools which provide support on how to design the appropriate primers for a given gene.

The detection of kynureninase expression levels takes place according to standard real time PCR protocols, e.g. with the FastLane Cell SYBR Green Kit provided by Qiagen. General technical information can furthermore be found in Logan & Saunders (2009).

After real time PCR has been completed, the Kynureninase expression level is compared to a house-keeping gene such as GAPFH to allow relative quantification. This method can be used to compare kynureninase level in physiologic vs pathologic situations.

### 11.2. In Situ Hybridization and In Situ PCR of Kynureninase

In situ hybridization (ISH) applies the methodology of the nucleic acid hybridization technique to the cellular level. Combining cytochemistry and immunocytochemistry, In Situ PCR allows the identification of cellular markers to be identified, and further permits the localization of to cell specific sequences within cell populations, such as tissues and blood samples.

In Situ PCR is limited to the detection of non-genomic material such as RNA, genes or genomes, as the detection limit in most conditions is several copies of the target nucleic acid per cell. Therefore, due to copy number limitations, hybridization of RNA is more sensitive than DNA detection.

In either case, at least one slice of a tissue of interest is provided, and in either case, sequence specific probes have to be synthesized which are able to hybridize with a nucleic acid encoding for kynureninase (either genomic DNA, mRNA or cDNA). Said probes may serve as primers of PCR and/or as labelling probes. Suitable protocols for in situ hybridization can be found in Braissant O, Wahli W (1998), and suitable protocols for in situ PCR can be found, e.g., in Nuovo (1995)

12. Detection of Enzymes and Metabolites of the Kynurenine Pathway with Other Methods.

Enzymes and metabolites of the kynurenine pathway can furthermore be detected with HPLC (high performance liquid chromatography), GC-MS (gas chromatography/mass spectrometry), and/or LC-MS (liquid chromatography/mass spectrometry). These methods are all within the scope of routine of the skilled person.

### REFERENCES MENTIONED IN THE TEXT

Bahr G M, Tello D S, Chedid L D. Marked enhancement in vivo of adjuvant activity of muramyl dipeptide to protein antigens and to synthetic weak immunogens with monoclonal anti-muramyl dipeptide antibodies. Infect Immun. 1985 August; 49(2): 312-319. PMCID: PMC262016
Lonberg N. Human antibodies from transgenic animals. Nat Biotechnol. 2005 September; 23(9): 1117-25.
Herren W. Simultaneous Humanization and Affinity Optimization of Monoclonal Antibodies. Springer Protocols 207, 197-212 DOI-10.1385/1-59259-334-8:197
Reynolds, J. C., S. DelVecchio, H. Sakahara, and M. Lora. 1989. Anti-murine response to mouse monoclonal antibodies. Nucl. Med. Biol. 16:121
Winter G., and W. J. Harris. 1993. Humanized antibodies. Immunol. Today 14:243.
Shultz L D, Ishikawa F, Greiner D L (2007). Humanized mice in translational biomedical research. Nat Rev Immunol 7:118-130.
Köhler G, Milstein, C (1975). Continuous cultures of fused cells secreting antibody of predefined specificity”. Nature 256 (5517): 495-497.
Brichta J, Hnilova M, Viskovic T: Generation of hapten-specific recombinant antibodies:antibody phage display technology: a review. Vet. Med.—Czech, 50, 2005 (6): 231-252
Kerrm Y. F Yaul, Hung Lee, J. Christopher Hall: Emerging trends in the synthesis and improvement of hapten-specific recombinant antibodies. Biotechnology Advances Volume 21, Issue 7, October 2003, Pages 599-637
Charlton Keith A., Porter Andrew J.: Isolation of Anti-Hapten Specific Antibody Fragments from Combinatorial Libraries. Springer Protocols (2001) 178, pp 159-171
Claudia Sheedy, C. Roger MacKenzie, J. Christopher Hall: Isolation and affinity maturation of hapten-specific antibodies. Biotechnology Advances Volume 25, Issue 4, July-August 2007, Pages 333-352
Pattyn, F., Speleman, F., De Paepe A. & Vandesompele, J. (2003). RTPrimerDB: the Real-Time PCR primer and probe database. Nucleic Acids Research, 31(1): 122-123.
Logan J, Edwards K, Saunders N (editors) (2009). Real-Time PCR: Current Technology and Applications. Caister Academic Press. ISBN 978-1-904455-39-4.
Nuovo G J (1995): In situ PCR: protocols and applications. Genome Res. 1995 (4), 151-167
Braissant O, Wahl W (1998): A Simplified In Situ Hybridization Protocol Using Non-radioactively Labeled Probes to Detect Abundant and Rare mRNAs on Tissue Sections Biochemica (1) 1998, 10-16
Hennie R Hoogenboom H R (2005): Selecting and screening recombinant antibody libraries. Nature Biotechnology 23, 1105-1116 (2005)
Inglese J, Shamu C E, Kiplin Guy R (2007) Reporting data from high-throughput screening of small-molecule libraries Nature Chemical Biology 3, 438-441 (2007)
Karpas A, Dremucheva A, Czepulkowski B H: A human myeloma cell line suitable for the generation of human monoclonal antibodies Proc. Natl. Acad. Sci. USA 13 Feb. 2001: 1799-1804.
Hanahan D, Weinberg R A. Hallmarks of cancer: the next generation. Cell 2011; 144:646-74.
Dunn G P, Bruce A T, Ikeda H, Old L J, Schreiber R D. Cancer immunoediting: from immunosurveillance to tumor escape. Nat Immunol 2002; 3:991-8.
Uyttenhove C, Pilotte L, Théate I, Stroobant V, Colau D, Parmentier N, et al. Evidence for a tumoral immune resistance mechanism based on tryptophan degradation by indoleamine 2,3-dioxygenase. Nat Med 2003; 9:1269-74.
Ferdinande L, Decaestecker C, Verset L, Mathieu A, Moles Lopez X, Negulescu A M, et al. Clinicopathological significance of indoleamine 2,3-dioxygenase 1 expression in colorectal cancer. Br J Cancer 2012; 106:141-7.
Pilotte L, Larrieu P, Stroobant V, Colau D, Dolusic E, Frederick R, et al. Reversal of tumoral immune resistance by inhibition of tryptophan 2,3-dioxygenase. Proc Natl Acad Sci USA 2012; 109:2497-502.
Opitz C A, Litzenburger U M, Sahm F, Ott M, Tritschler I, Trump S, et al. An endogenous tumour-promoting ligand of the human aryl hydrocarbon receptor. Nature 2011; 478:197-203.
Muller A J, DuHadaway J B, Donover P S, Sutanto-Ward E, Prendergast G C. Inhibition of indoleamine 2,3-dioxygenase, an immunoregulatory target of the cancer suppression gene Bin1, potentiates cancer chemotherapy. Nat Med 2005; 11:312-9.
Köhler G, Milstein C. Continuous cultures of fused cells secreting antibody of predefined specificity. Nature 1975; 256:495-7.
Prendergast G C. Immune escape as a fundamental trait of cancer: focus on IDO. Oncogene 2008; 27:3889-900.

### REFERENCES MENTIONED IN TABLE 1

16. Uyttenhove C, Pilotte L, Théate I, Stroobant V, Colau D, Parmentier N, et al. Evidence for a tumoral immune resistance mechanism based on tryptophan degradation by indoleamine 2,3-dioxygenase. Nat Med 2003; 9:1269-74.
17. Ferdinande L, Decaestecker C, Verset L, Mathieu A, Moles Lopez X, Negulescu A M, et al. Clinicopathological significance of indoleamine 2,3-dioxygenase 1 expression in colorectal cancer. Br J Cancer 2012; 106:141-7.
19. Opitz C A, Litzenburger U M, Sahm F, Ott M, Tritschler I, Trump S, et al. An endogenous tumour-promoting ligand of the human aryl hydrocarbon receptor. Nature 2011; 478:197-203.
23. Nakamura T, Shima T, Saeki A, Hidaka T, Nakashima A, Takikawa O, Saito S. Expression of indoleamine 2,3-dioxygenase and the recruitment of Foxp3-expressing regulatory T cells in the development and progression of uterine cervical cancer. Cancer Sci 2007; 98:874-81.
24. Brandacher G, Perathoner A, Ladurner R, Schneeberger S, Obrist P, Winkler C, et al. Prognostic value of indoleamine 2,3-dioxygenase expression in colorectal cancer: effect on tumor-infiltrating T cells. Clin Cancer Res 2006; 12:1144-51.
25. Ino K, Yoshida N, Kajiyama H, Shibata K, Yamamoto E, Kidokoro K, et al. Indoleamine 2,3-dioxygenase is a novel prognostic indicator for endometrial cancer. Br J Cancer 2006; 95:1555-61.
26. Pan K, Wang H, Chen M S, Zhang H K, Weng D S, Zhou J, et al. Expression and prognosis role of indoleamine 2,3-dioxygenase in hepatocellular carcinoma. J Cancer Res Clin Oncol 2008; 134:1247-53.
27. Curti A, Trabanelli S, Onofri C, Aluigi M, Salvestrini V, Ocadlikova D, et al. Indoleamine 2,3-dioxygenase-expressing leukemic dendritic cells impair a leukemia-specific immune response by inducing potent T regulatory cells. Haematologica 2010; 95:2022-30.
28. Brody J R, Costantino C L, Berger A C, Sato T, Lisanti M P, Yeo C J, et al. Expression of indoleamine 2,3-dioxygenase in metastatic malignant melanoma recruits regulatory T cells to avoid immune detection and affects survival. Cell Cycle 2009; 8:1930-4.
29. Liu P, Xie B L, Cai S H, He Y W, Zhang G, Yi Y M, Du J. Expression of indoleamine 2,3-dioxygenase in nasopharyngeal carcinoma impairs the cytolytic function of peripheral blood lymphocytes. BMC Cancer 2009; 9:416.
30. Inaba T, Ino K, Kajiyama H, Yamamoto E, Shibata K, Nawa A, et al. Role of the immunosuppressive enzyme indoleamine 2,3-dioxygenase in the progression of ovarian carcinoma. Gynecol Oncol 2009; 115:185-92.
31. Witkiewicz A K, Costantino C L, Metz R, Muller A J, Prendergast G C, Yeo C J, Brody J R. Genotyping and expression analysis of IDO2 in human pancreatic cancer: a novel, active target. J Am Coll Surg 2009; 208:781-7; discussion 787-9.
32. Astigiano S, Morandi B, Costa R, Mastracci L, D'Agostino A, Ratto G B, et al. Eosinophil granulocytes account for indoleamine 2,3-dioxygenase-mediated immune escape in human non-small cell lung cancer. Neoplasia 2005; 7:390-6.
33. Mansfield A S, Heikkila P S, Vaara A T, von Smitten K A, Vakkila J M, Leidenius M H. Simultaneous Foxp3 and IDO expression is associated with sentinel lymph node metastases in breast cancer. BMC Cancer 2009; 9:231.

1-37. (canceled)

38. A monoclonal antibody or an antigen-binding fragment thereof, wherein the antibody is directed against a metabolite of the kynurenine pathway, said metabolite is selected from the group consisting of 3-Hydroxy Anthranilic Acid (3HAA), Kynurenine, Cinnabarinic Acid, and Quinolinic Acid.

39. A method of using the antibody or the antigen binding fragment of claim 38, the method comprising obtaining a biological sample and contacting said biological sample with said antibody or antigen biding fragment thereof, thereby determining the amount of the metabolite in the biological sample.

40. A method of treating a neoplastic disease in a human or animal patient in need of such treatment, said method comprising:
administering to the patent a modulator of the kynurenine pathway,
thereby reducing the formation, concentration, availability, or effect of at least one of: 3-Hydroxy Anthranilic Acid (3HAA), L-Kynurenine, Quinolinic Acid, Cinnabarinic Acid, and Kynureninase.

41. The method of claim 40, wherein the modulator is an antagonist to an enzyme and/or a metabolite of the kynurenine pathway.

42. The method of claim 41, wherein said enzyme of the kynurenine pathway is at least one selected from the group consisting of Kynurenine formamidase, Kynurenine amino-transferase, Kynurenine 3-hydroxylase (also called Kynurenine mono-oxygenase), Kynureninase (also called L-Kynurenine hydrolase), Kynurenine amino-transferase, and 3-Hydroxyanthranilic Acid oxygenase (also called 3-Hydroxyanthranilate dioxygenase).

43. The method of claim 41, wherein the antagonist is selected from the group consisting of a monoclonal antibody, a fragment or a derivative of a monoclonal antibody, a fusion peptide comprising at least one domain capable of binding an enzyme and/or a metabolite of the kynurenine pathway, an antibody mimetic, an aptamer, and a small molecule antagonist.

44. The method of claim 43, wherein the monoclonal antibody is murine, chimeric, humanized, or human.

45. The method of claim 41, wherein the antagonist is a monoclonal antibody or an antigen-binding fragment thereof or an antibody mimetic is directed against Kynurenine, Cinnabarinic Acid, or Quinolinic Acid.

46. The method of claim 41, wherein a neoplastic disease is a cancer characterized by overexpression of Indoleamine 2,3-dioxygenase 1 (IDO1), Indoleamine 2,3 dioxygenase 2 (IDO2), and/or Tryptophan 2,3-dioxygenase (TDO2).

47. The method of claim 46, wherein a neoplastic disease is a cancer characterized by overexpression of IDO1 and/or TDO2.

48. The method of claim 41, wherein a neoplastic disease is selected from the group consisting of colorectal cancer, breast cancer, melanoma, glioma, and a cancer of the central nervous system.

49. The method of claim 41, the method further comprising administering to the patient a second treatment selected from the consisting of a antineoplastic agent, a targeted drug, an endocrine drug, a tumor vaccine, immunotherapy, a cellular therapy, radiotherapy, surgery, and laser ablation.

50. A method of diagnosis, prognosis, risk assessment, and/or prediction of a neoplastic disease in a human or animal subject, said method comprising:
providing a biological sample,
having determined in said sample the presence and/or concentration of an enzyme and/or a metabolite of the kynurenine pathway,
optionally, having determined in the sample the expression level(s) of Indoleamine 2,3-dioxygenase 1 (IDO1), Indoleamine 2,3 dioxygenase 2 (IDO2), and/or Tryptophan 2,3-dioxygenase (TDO2),
wherein elevated level(s) of said enzyme or metabolite of the kynurenine pathway indicate(s) that subject has or is at risk for developing a neoplastic disorder and may benefit from a treatment by a modulator of the kynurenine pathway, and
optionally, receiving a treatment comprising the method of claim 40.

51. The method of claim 50, where said levels in the biological sample are determined by immunohistochemistry, ELISA, EIA and/or immunofluorescence, in situ PCR in tissue slices, RealTime PCR, gas chromatography/mass spectroscopy (GC/MS), high performance liquid chromatography (HPLC), or liquid chromatography/mass spectroscopy (LC/MS).

52. The method of claim 50, said method comprising:
having determined the sample the expression level of Indoleamine 2,3-dioxygenase 1 (IDO1), Indoleamine 2,3 dioxygenase 2 (IDO2), and/or Tryptophan 2,3-dioxygenase (TDO2).

53. The method of claim 50, said method comprising:
receiving a treatment comprising the method of claim 40.

54. The method of claim 50, said method comprising:
having determined in the sample the expression level of Indoleamine 2,3-dioxygenase 1 (IDO1), Indoleamine 2,3 dioxygenase 2 (IDO2), and/or Tryptophan 2,3-dioxygenase (TDO2), and receiving a treatment comprising the method of claim 40.
